(19) United States
(12) Patent Application Publication
(10) Pub. No.: US 2011/0092591 A1
(43) Pub. Date: Apr. 21, 2011

(54) PHENYL-PRENYL DERIVATIVES, OF MARINE AND SYNTHETIC ORIGIN, FOR THE TREATMENT OF COGNITIVE, NEURODEGENERATIVE OR NEURONAL DISEASES OR DISORDERS

(75) Inventors: Javier Lopez Ogalla, Madrid (ES); Pilar Munoz Ruiz, Madrid (ES); Diana Alonso Gordillo, Madrid (ES); Miguel Medina Padilla, Madrid (ES); Esther Garcia Palomero, Madrid (ES); Ana Martinez Gil, Madrid (ES); Ana Castro Morera, Madrid (ES)

(73) Assignee: NOSCIRA S.A., Tres Cantos - Madrid (ES)

(21) Appl. No.: 12/866,085

(22) PCT Filed: Feb. 6, 2009
(86) PCT No.: PCT/EP2009/051373

§ 371 Date: Jan. 4, 2011

(30) Foreign Application Priority Data
Feb. 6, 2008 (EP) 08380031.8

(51) Int. Cl. A61K 31/235; C07C 65/01; C07C 69/86; C07C 233/65; C07C 69/618; C07C 47/565; A61K 31/192; A61K 31/166; A61K 31/11; A61P 25/28; A61P 25/16; A61P 29/00; A61P 25/08

(52) U.S. Cl. 514/533; 562/475; 560/72; 564/177; 560/144; 568/442; 514/568; 514/543; 514/622; 514/701

(57) ABSTRACT

The present invention is related to a family of phenyl-prenyl derivatives of formula (I), and to their use in the treatment of cognitive, neurodegenerative or neuronal diseases or disorders, such as Alzheimer's disease or Parkinson's Disease. The present invention also relates to pharmaceutical compositions comprising the same. Further, the present invention is directed to the compounds of formula (I) for medical use, particularly for the use for the treatment and/or prevention of a cognitive, neurodegenerative or neuronal disease or disorder, and to the use of the compounds in the manufacture of a medicament for the treatment and/or prevention of a cognitive, neurodegenerative or neuronal disease or disorder.

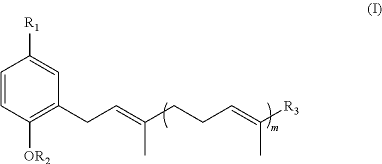

### FIELD OF THE INVENTION

The present invention is related to a family of phenyl-prenyl derivatives of formula (I), and to their use in the treatment of cognitive, neurodegenerative or neuronal diseases or disorders, such as Alzheimer's disease or Parkinson's Disease. The present invention also relates to pharmaceutical compositions comprising the same. Further, the present invention is directed to the use of the compounds in the manufacture of a medicament for the treatment and/or prevention of a cognitive, neurodegenerative or neuronal disease or disorder.

### BACKGROUND OF THE INVENTION

Glycogen synthase kinase 3 (GSK-3) is a serine-threonine protein kinase comprised of α and β isoforms which phosphorylates diverse target proteins, such as enzymes or transcription factors. GSK-3β plays an important regulatory role in several signaling pathways of cellular processes, such as initiation of protein synthesis, cell proliferation, apoptosis or embryonic development (Discovery and development of GSK3 inhibitors for the treatment of type 2 diabetes, Wagman et al., Curr. Pharm. Des. 2004; 10(10):1105-37). Disorders in many of these regulatory pathways are involved in human diseases, such as Parkinson's Disease (GSK-3beta inhibition/beta-catenin stabilization in ventral midbrain precursors increases differentiation into dopamine neurons, Castelo-Branco et al., J Cell Sci. 2004 Nov. 15; 117(Pt 24):5731-7), Alzheimer's Disease, type II diabetes, bipolar disorders, diseases caused by unicellular parasites that express GSK3 homologues (Pharmacological inhibitors of glycogen synthase kinases 3, Maijer L et al., Trends Pharmacol. Sci. 2004; 25(9):471-80)) or prion-induced neurodegeneration (Prion peptide induces neuronal cell death through a pathway involving glycogen synthase kinase 3, Perez M. et al., Biochem. J. 2003; 372(Pt 1): 129-36).

An important regulatory process wherein GSK-3 takes part is the Wnt pathway. The Wnts are a family of cysteine-rich and glycosylated proteins which act as activators of different processes, such as cell growth differentiation, migration and fate (The Wnts, Miller J R, Genome Biol. 2002; 3(1):REVIEWS3001). A key protein of this pathway is the β-catenin, which translocates to the nucleus and activates different genes when a Wnt binds to its receptor. A multi protein complex which includes APC (adenomatous polyposis coli) and axin, among other proteins, facilitates that GSK-3 phosphorilates β-catenin in several sites of its N-terminal domain. This event triggers the binding of ubiquitin to the phosphorylated β-catenin and its subsequent degradation in the proteasome.

Alzheimer's Disease (AD) is a neurodegenerative disorder characterized by the presence of β-Amyloid protein deposits in the core of neuritic plaques and abnormal neurofibrillary tangles in the brain of AD patients. The Amyloid β-protein (Aβ) is formed by two endoproteolytic cleavages of the Amyloid β protein precursor (AβPP), a large transmembrane type I protein. A protease termed β-secretase cleaves AβPP at the N-terminus of the Aβ domain to generate the soluble AβPP and the membrane anchored C-terminal fragments (CTFs). Then, a second secretase called γ-secretase, cuts CTFs within the transmembrane region to form Aβ, which is secreted from the cells. The identification of compounds able to prevent or reduce this event has become an important goal for the research on the treatment of AD.

Also other diseases have been linked to the presence of beta Amyloid deposits in the brain. Some examples are MCI (mild cognitive impairment), Down's syndrome, Hereditary Cerebral Hemorrhage with Amyloidosis of the Dutch-Type, cerebral Amyloid angiopathy, other degenerative dementias, including dementias of mixed vascular and degenerative origin, dementia associated with Parkinson's disease, dementia associated with progressive supranuclear palsy, dementia associated with cortical basal degeneration, and diffuse Lewy body type Alzheimer's disease (see publication US20040132782).

BACE (β site APP cleaving enzyme) is an aspartyl protease with β-secretase activity. BACE is a type I integral membrane protein with a typical aspartyl protease motif in its luminal domain. BACE hydrolyzes AβPP specifically at the Met-Asp site, with an acidic pH optimum. BACE is highly expressed in the brain and it colocalizes with the intracellular sites of CTFs and Aβ production. BACE has become an important target for the development of therapeutic compounds against Alzheimer's Disease.

There are several factors that increase the expression and activity of BACE. Oxidant agents and oxidative products, such as H₂O₂ or HNE (4-hydroxynonenal), which is an aldehydic end product of polyunsaturated fatty acids, were shown to increase intracellular and secreted Aβ levels in neuronal and non neuronal cells (Paola et al. 2000; Misonou et al. 2001; Frederikse et al. 1996). Many studies have been carried out to determine the cellular mechanisms that underlie the Aβ overproduction. In 2002, Tamagno et al. (Oxidative Stress Increases Expression and Activity of BACE in NT₂ Neurons, 2002, Neurobiol. Dis., 10, 279-288) demonstrated that oxidative stress induces BACE protein levels and activity, and this event is mediated by the oxidative product HNE. According to this study, exposure of NT₂ cells to oxidant agents did not influence AβPP expression. The effect of these agents on Aβ is related to an increase of BACE1 expression via transcriptional up regulation of BACE1 gene (Oxidative stress potentiates BACE1 gene expression and Aβ generation, Tong et al., 2004, J. Neural. Transm., 112(3):455-69).

The identification of compounds which are able to prevent the effect of oxidative agents has become an important goal of current research in Alzheimer's Disease. Among these compounds, dehydroepiandrosterone (DHEA) and its role in the CNS have been studied by Tamagno et al. (Dehydroepiandrosterone reduces expression and activity of BACE in NT₂ neurons exposed to oxidative stress, Tamagno et al., 2003, Neurobiol. Dis., 14, 291-301). DHEA is an adrenal steroid that serves as a precursor to both androgens and estrogens and is synthesized from sterol precursors in the nervous system (Balieu 1981). DHEA is known to improve a variety of functional activities in the CNS, including increased memory and learning in different animal models (Vallée et al. 2001) and exerts protection against excitatory amino acids and Aβ neurotoxicity. In this study, it has been demonstrated that a pre-treatment with DHEA is able to decrease the expression, protein levels and activity of BACE induced in NT₂ neurons by oxidative agents, such as Asc/Fe and H₂O₂/Fe. This protection seems to be due to the antioxidant properties of the steroid, able to prevent the production of the end products of lipid oxidation, such as HNE. The oxidative stress products induce an increase of BACE protein levels and activity, and this induction is due to a gene overexpression, as has been demonstrated by quantitative PCR analysis. Decline of DHEA concentrations with ageing led to the suggestion that it could be implicated in longevity and that its progressive decrease can be related with some of the aging-related degenerative disorders, including AD. In conclusion, DHEA is able to prevent the oxidative stress-dependent Amyloidogenic processing of AβPP through the negative modulation of the expression and activity of BACE.

U.S. Pat. No. 6,001,880 discloses pirazoline derivatives useful as radical scavengers. As intermediates for the synthesis of said pirazoline derivatives 3,4-digeranyloxibenzoic acid and ethyl 3,4-digeranyloxibenzoate are disclosed. No mention is made of their usefulness in the treatment of cognitive, neurodegenerative or neuronal diseases or disorders.

In Chemical Abstract (accession number 2001:184028) it is disclosed that ethyl 4-hydroxy-3-prenyloxybenzoic acid is useful in the 3D-HPLC analysis. No mention is made of its usefulness in the treatment cognitive, neurodegenerative or neuronal diseases or disorders.

Baek, S. H., et al, J. of Nat. Prod., 1998, 1143-1145 discloses compounds with cytotoxic activity. As intermediates in the synthesis of said compounds methyl 3,4-digeranyloxybenzoate, methyl 4-hydroxy-3-geranyloxybenzoate, and methyl 4-methoxy-3-geranyloxybenzoate are mentioned. No mention is made of any therapeutic activity of said synthetic intermediates.

EP 0 869 118 discloses antibacterial activity of pyrrolidine derivatives. As intermediates for the synthesis of said pyrrolidine derivatives 3,4-prenyloxybenzoic acid, 3,4-geranyloxybenzoic acid and 4-methoxy-3-geranyloxybenzoic acid are disclosed. No mention is made of their usefulness in the treatment of cognitive, neurodegenerative or neuronal diseases or disorders.

WO 94/02465 Å discloses compounds for inhibiting tumour necrosis factor. 4-methoxy-3-prenyloxybenzoic acid is disclosed as a synthetic intermediate of the active compounds. No mention is made of any therapeutic activity of said synthetic intermediate.

The expression of BACE has been localized in the brain, in particular in neurons, indicating that neurons are the major source of β-Amyloid peptides in the brain. Astrocytes, on the other hand, are known to be important for β-Amyloid clearance and degradation, for providing trophic support to neurons and for forming a protective barrier between β-Amyloid deposits and neurons. However, according to Rossner et al. (Alzheimer's disease β-secretase BACE1 is not a neuron specific enzyme, Rossner et al., J Neurobiochem. 2005, 92, 226-234), astrocytes may also represent an alternative cellular source of β-Amyloid peptides. The role of astrocytes in the pathogenesis of AD remains undetermined and may differ on a case to case instance due to dependence on a broad spectrum of interactive events in neurons, astrocytes and microglia.

### SUMMARY OF THE INVENTION

It has been found that organic solvent extracts of the marine sponge Sarcotragus showed interesting biological activity, namely as GSK-3 inhibitors, as well as BACE inhibitors. Fractionation and purification of the active components from these extracts resulted in the isolation of a series of phenyl-prenyl compounds, with a potential use as therapeutic agents. Further details are given in the examples of the present specification. Synthetic derivatives have been designed to improve the properties of the original compounds.

Therefore, the present invention is related to a new family of phenyl-prenyl derivatives of general formula (I). They have shown to exhibit an inhibitory effect on the enzymatic targets GSK-3, and most of them also on BACE, in in vitro assays. GSK-3, as detailed above, is known to play an important role in numerous diseases and conditions of very diverse nature, specially cognitive, neurodegenerative or neuronal diseases, and thus the inhibition of this enzyme is known to be a good therapeutic approach for the treatment of said diseases and conditions. Further, the inhibition of BACE enzyme, as detailed above, is also a good therapeutic target for the treatment of a number of diseases and conditions. Thus, taking into account that these enzymes are known to be involved in a variety of cognitive, neurodegenerative or neuronal diseases or disorders, and that their inhibition is known to help to prevent and treat these diseases, the compounds of formula (I) are useful for the prevention and/or treatment of cognitive, neurodegenerative or neuronal diseases or disorders.

Therefore, in a first aspect, the present invention is related to a novel compound of formula (I) (also referred to as the compound of the invention)

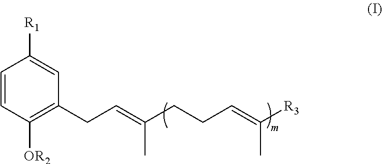

wherein
m is an integer selected from 0, 1, 2, 3, 4, and 5;
R₁ is selected from —C(═O)OR₄, —CHO and —CONH—R₅,
wherein R₄ is selected from hydrogen, —CH₂-Ph, —CH₂—O—CH₃,
and R₅ is C₁-C₆ alkyl
R₂ is selected from hydrogen, phenyl, benzyl, —COR₆ and —CH₂—O—CH₃,
wherein R₆ is selected from hydrogen and C₁-C₆ alkyl,
R₃ is selected from —CH₃ and

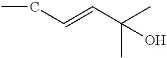

and salts, preferably any pharmaceutically acceptable salts, solvates and prodrugs thereof.

The compounds of formula (I) may comprise asymmetric substituents, i.e. asymmetric substituents in R₁ and/or R₂, which may give raise to the presence of different stereoisomers (enantiomers, diastereoisomers, etc). The present invention comprises all such stereoisomers.

A further aspect of the present invention is a novel compound of formula (I) as defined above, for use as a medicament.

The present invention is further related to a pharmaceutical composition comprising at least one of the compounds of formula (I) as defined above, or salts, solvates or prodrugs thereof, and at least one pharmaceutically acceptable carrier, adjuvant and/or vehicle.

The compounds of formula (I) are prepared according to the following procedure:

3-bromo-4-hydroxybenzaldehyde (A), a commercially available compound, is used as starting reactive, which is protected in the form of an acetal; for this purpose, Ethylene glycol and p-Toluenesulfonic acid monohydrate are added, thus obtaining the protected aldehyde (B). The protection of the phenolic alcohol was performed adding Methyl chloromethyl ether together with DIPEA (Diisopropyl ethylamine) in THF (Tetrahydrofuran), obtaining the protected phenol (C) (see scheme 1).

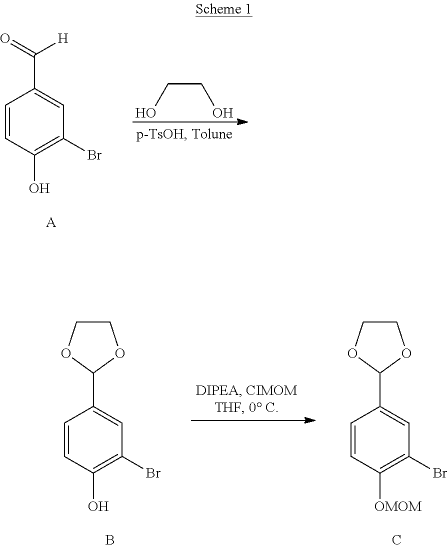

Once the aldehyde and the phenol are protected, an alkylation reaction is performed, using as alkylating agents the prenylic chains of (2E)-1-bromo-3,7-dimethyl-2,6-octadiene (D), (2E,6E)-1-bromo-3,7,11-trimethyl-2,6,10-dodecatriene (E), both of them commercially available, and geranylgeranyl bromide, which was obtained starting from 3,7,11,15-Tetramethyl-1,6,10,14-hexadecatetraen-3-ol (F), using PBr₃ (Phosphorus(III) bromide) in ethylic ether, thus obtaining product G (see scheme 2).

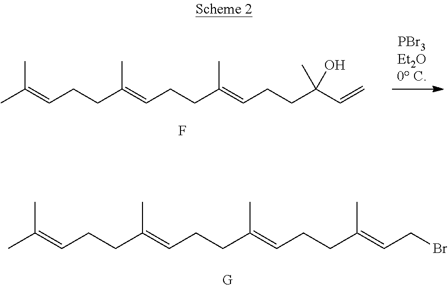

In all the cases the alkylation was performed using product C, to which a solution of n-BuLi (Lithium-1-butanide) was added, together with CuBr.DMS (Copper (I) bromide-dimethyl sulfide complex), and the corresponding prenylic bromide, in a mixture of toluene and anhydrous ethylic ether in a relation of 1:1, thus obtaining the corresponding aldehydes 8-10 (see scheme 3).

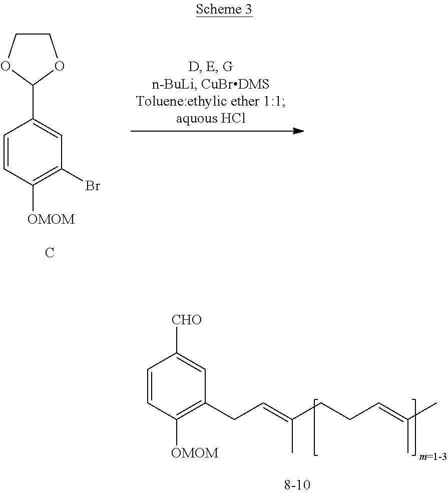

The subsequent deprotection of the methoxymethyl ether using CSA ((±)-Camphor-10-sulfonic acid) in methanol provided the corresponding alcohols 11-13. The oxidation of the aldehyde was performed using NaH₂PO₄ (Sodium dihydrogen phosphate) and NaClO₂ (Sodium chlorite) in a mixture of THF/water in a relation of 1:4, providing the acids 14-16 (see scheme 4).

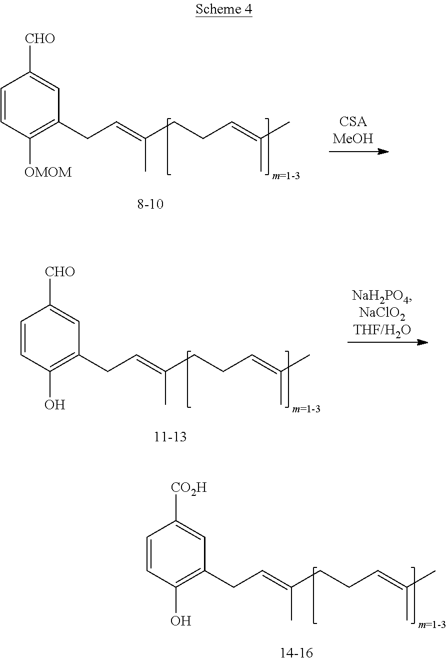

Starting from product 16, compounds with m=3 where obtained. The reaction of product 16 with BrBn (Benzyl Bromide), in the presence of K₂CO₃ (Potassium carbonate) in DMF (N,N-Dimethylformamide) provided product 17 (see scheme 5).

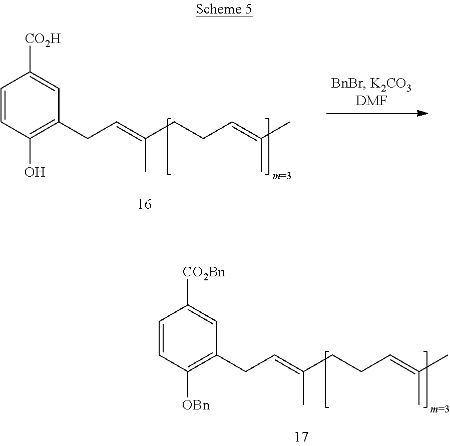

In order to obtain the corresponding amide (18), Ethylamine was used, together with EDC (N-(3-Dimethylaminopropyl)-W-ethylcarbodiimide) and HOBt (1-Hydroxybenzotriazole) in Dichloromethane (see scheme 6).

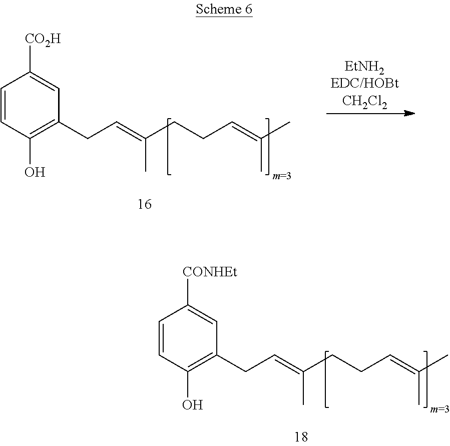

Product 16 was reacted with Acetic anhydride in Pyridine, obtaining the protected product 19 with a yield of 100%. The subsequent reaction with Methyl chloromethyl ether together with DIPEA (Diisopropyl ethylamine) in THF (Tetrahydrofuran) provided the product 20 with a yield of 75% (see scheme 7).

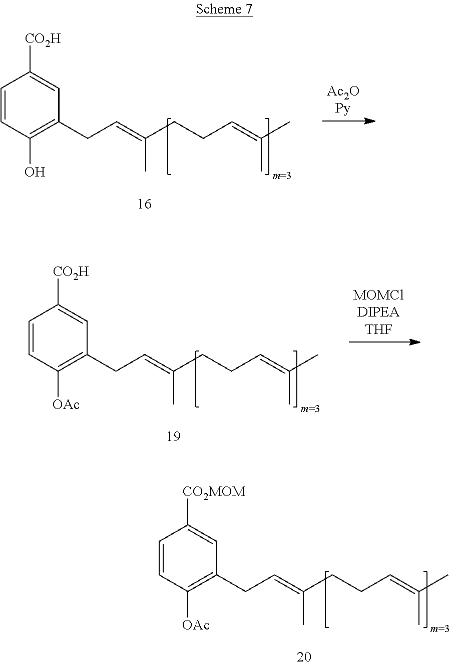

Another aspect of the present invention is the use of a compound of formula (I*)

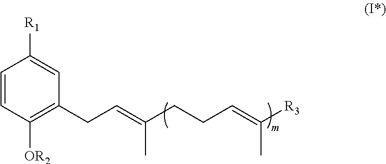

wherein
m is an integer selected from 0, 1, 2, 3, 4, and 5;
R₁ is selected from C₁-C₁₂ alkoxy, —CH₂—O—CH₃, —OH, —C(═O)OR₄, —CHO and —CONH—R₅,
wherein R₄ is selected from hydrogen, C₁-C₆ alkyl, —CH₂-Ph, —CH₂—O—CH₃,
and R₅ is C₁-C₆ alkyl,
R₂ is selected from hydrogen, phenyl, benzyl, —COR₆, C₁-C₆ alkyl and —CH₂—O—CH₃,
wherein R₆ is selected from hydrogen and C₁-C₆ alkyl,
R₃ is selected from —CH₃ and

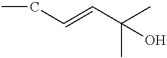

and salts, preferably any pharmaceutically acceptable salts, solvates and prodrugs thereof;
in the manufacture of a medicament for the treatment and/or profilaxis of a cognitive, neurodegenerative or neuronal disease or disorder.

A further aspect is a compound of formula (I*) for use in the treatment and/or profilaxis of a cognitive, neurodegenerative or neuronal disease or disorder.

In a further aspect, the present invention is related to a method of treating and/or preventing a cognitive, neurodegenerative or neuronal disease or disorder, which method comprises administering to a patient in need of such a treatment a therapeutically effective amount of at least one compound of formula (I*) as defined in above or a pharmaceutical composition thereof.

### DETAILED DESCRIPTION OF THE INVENTION

In the above definition of compounds of formula (I) the following terms have the meaning indicated:

The term “C₁-C₁₂ alkyl” refers to a linear or branched hydrocarbon chain radical consisting of carbon and hydrogen atoms, containing no unsaturation, having one to twelve, preferably one to six (“C₁-C₆ alkyl”), carbon atoms, and which is attached to the rest of the molecule by a single bond. Examples of alkyl groups include, but are not limited to alkyl groups such as methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl. An alkyl group can be unsubstituted or substituted with one or two suitable substituents as described below.

References herein to substituted groups in the compounds of the present invention refer to the specified moiety that may be substituted at one or more available positions by one or more suitable groups, e.g., halogen such as fluoro, chloro, bromo and iodo; cyano; hydroxyl; nitro; azido; alkanoyl such as a C₁₋₆ alkanoyl group such as acyl and the like; carboxamido; alkyl groups including those groups having 1 to about 12 carbon atoms or from 1 to about 6 carbon atoms and more preferably 1-3 carbon atoms; alkenyl and alkynyl groups including groups having one or more unsaturated linkages and from 2 to about 12 carbon or from 2 to about 6 carbon atoms; alkoxy groups having one or more oxygen linkages and from 1 to about 12 carbon atoms or 1 to about 6 carbon atoms; aryloxy such as phenoxy; alkylthio groups including those moieties having one or more thioether linkages and from 1 to about 12 carbon atoms or from 1 to about 6 carbon atoms; alkylsulfinyl groups including those moieties having one or more sulfinyl linkages and from 1 to about 12 carbon atoms or from 1 to about 6 carbon atoms; alkylsulfonyl groups including those moieties having one or more sulfonyl linkages and from 1 to about 12 carbon atoms or from 1 to about 6 carbon atoms; aminoalkyl groups such as groups having one or more N atoms and from 1 to about 12 carbon atoms or from 1 to about 6 carbon atoms; carbocylic aryl having 6 or more carbons, particularly phenyl or naphthyl and aralkyl such as benzyl. Unless otherwise indicated, an optionally substituted group may have a substituent at each substitutable position of the group, and each substitution is independent of the other.

The term “C₂-C₁₂ alkenyl” means a linear or branched hydrocarbon chain radical having one or more carbon-carbon double bonds therein and having from two to twelve, preferably one to six (“C₁-C₆ alkenyl”), carbon atoms, and which is attached to the rest of the molecule by a single bond. The double bond of an alkenyl group can be unconjugated or conjugated to another unsaturated group. Suitable alkenyl groups include, but are not limited to alkenyl groups such as vinyl, allyl, butenyl (e.g. 1-butenyl, 2-butenyl, 3-butenyl), pentenyl (e.g. 1-pentenyl, 2-pentenyl, 3-pentenyl, 4-pentenyl), hexenyl (e.g. 1-hexenyl, 2-hexenyl, 3-hexenyl, 4-hexenyl, 5-hexenyl), butadienyl, pentadienyl (e.g. 1,3-pentadienyl, 2,4-pentadienyl), hexadienyl (e.g. 1,3-hexadienyl, 1,4-hexadienyl, 1,5-hexadienyl, 2,4-hexadienyl, 2,5-hexadienyl), 2-ethylhexenyl (e.g. 2-ethylhex-1-enyl, 2-ethylhex-2-enyl, 2-ethylhex-3-enyl, 2-ethylhex-4-enyl, 2-ethylhex-5-enyl), 2-propyl-2-butenyl, 4,6-Dimethyl-oct-6-enyl. An alkenyl group can be unsubstituted or substituted with one or two suitable substituents as described below.

The term “C₁-C₁₂ alkoxy” refers to a radical of the formula —ORa, wherein Ra is an alkyl radical as defined above, e.g., methoxy, ethoxy, propoxy, etc.

The term “alkoxymethyl ether” refers to a radical of formula —CH₂—O—R′, wherein R′ is an alkyl, alkenyl, aryl, aralkyl or trialkylsilyl radical as defined herein, such as methoxymethyl ether, 2-methoxyethoxymethyl ether, benzyloxymethyl ether, p-methoxybenzyloxymethyl ether, 2-(trimethylsilyl)ethoxymethyl ether.

The term “C₂-C₁₂ alkynyl” means a linear or branched hydrocarbon chain radical having one or more carbon-carbon triple bonds therein and from two to twelve, preferably one to six (“C₁-C₆ alkynyl”), carbon atoms, and which is attached to the rest of the molecule by a single bond. The triple bond of an alkynyl group can be unconjugated or conjugated to another unsaturated group. Suitable alkynyl groups include, but are not limited to alkynyl groups such as ethynyl, propynyl (e.g. 1-propynyl, 2-propynyl), butynyl (e.g. 1-butynyl, 2-butynyl, 3-butynyl), pentynyl (e.g. 1-pentynyl, 2-pentynyl, 3-pentynyl, 4-pentynyl), hexynyl (e.g. 1-hexynyl, 2-hexynyl, 3-hexynyl, 4-hexynyl, 5-hexynyl), methylpropynyl, 3-methyl-1-butynyl, 4-methyl-2-heptynyl, and 4-ethyl-2-octynyl. An alkynyl group can be unsubstituted or substituted with one or two suitable substituents as described below.

The term “C₁-C₁₂ alkylamino” is intended to mean “C₁-C₁₂ monoalkylamino”, and refers to an amino group attached to the rest of the molecule by a single bond, substituted with a single alkyl chain as defined above.

The term “C₁-C₁₂ dialkylamino” refers to an amino group attached to the rest of the molecule by a single bond, substituted with two alkyl chains, each one the same or different as defined above.

According to a first aspect, the present invention is related to a novel compound of general formula (I)

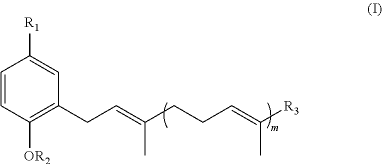

wherein
m is an integer selected from 0, 1, 2, 3, 4, and 5;
R₁ is selected from —C(═O)OR₄, —CHO and —CONH—R₅,
wherein R₄ is selected from hydrogen, —CH₂-Ph, —CH₂—O—CH₃,
and R₅ is C₁-C₆ alkyl,
R₂ is selected from hydrogen, phenyl, benzyl, —COR₆ and —CH₂—O—CH₃,
wherein R₆ is selected from hydrogen and C₁-C₆ alkyl.
R₃ is selected from —CH₃ and

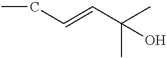

and salts, preferably any pharmaceutically acceptable salts, solvates and prodrugs thereof.

In order to clarify the meaning of m, it is indicated that when m is 0, compound of formula (I) is:

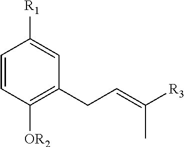

When m is 1, compound of formula (I) is:

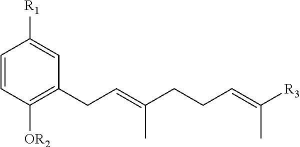

When m is 2, compound of formula (I) is:

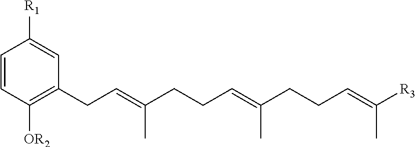

and so further.

According to an embodiment, m is selected from 0, 1, 2 and 3.

A preferred group of compounds of formula (I) are those wherein R₁ is —C(═O)OR₄, R₄ being selected from hydrogen, —CH₂—O—CH₃ and —CH₂-Ph. According to a still further preferred embodiment, R₄ is selected from —CH₂—O—CH₃ and —CH₂-Ph.

A further group of preferred compounds are those wherein R₁ is —CONH—R₅, R₅ being selected from methyl and ethyl.

According to another preferred embodiment, R₂ is selected from hydrogen, benzyl, —COCH₃ and —CH₂—O—CH₃. In a still further preferred embodiment, R₂ is selected from benzyl and —CH₂—O—CH₃.

A preferred group of compounds are those wherein R₃ is

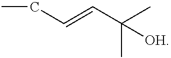

A further group of preferred compounds are those wherein m is an integer selected from 1, 2, 3, 4, and 5; R₁ is —CHO and R₂ is —CH₂—O—CH₃.

According to another preferred embodiment, m is an integer selected from 0, 1 and 2; R₁ is —C(═O)OH and R₂ is CH₂—O—CH₃.

A further group of preferred compounds are those wherein m is an integer selected from 2, 3, 4, and 5; R₁ is —CHO and R₂ is hydrogen.

According to another preferred embodiment, m is an integer selected from 2, 4 and 5; R₁ is —C(═O)OH and R₂ is hydrogen.

Preferred compounds of formula (I) are the following:

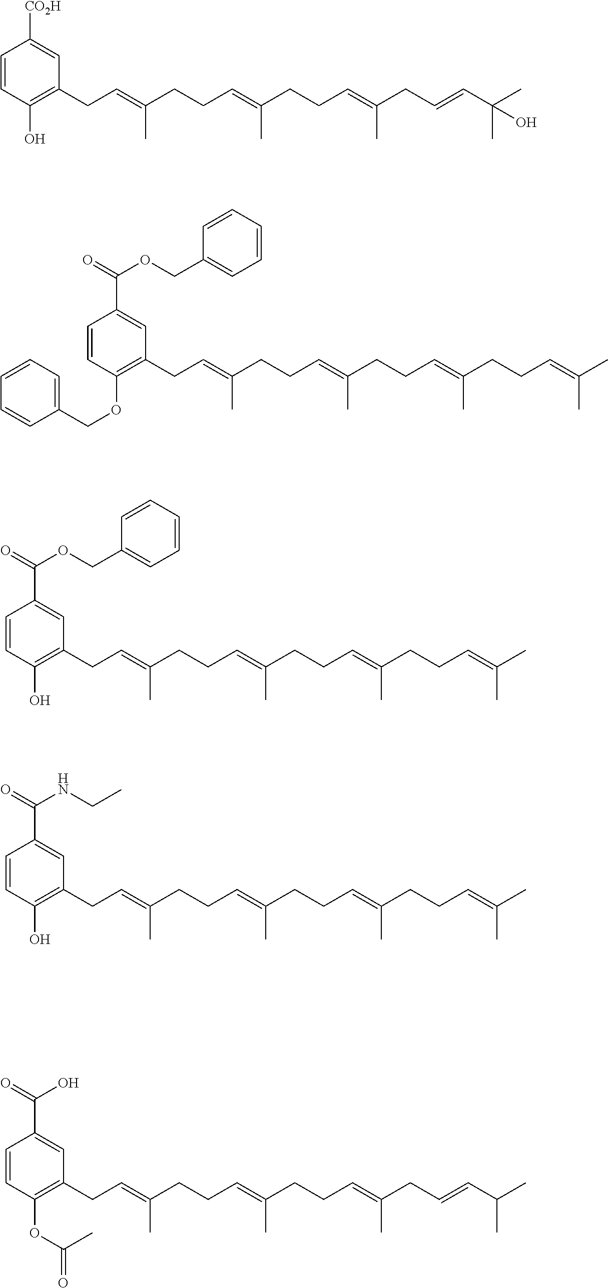

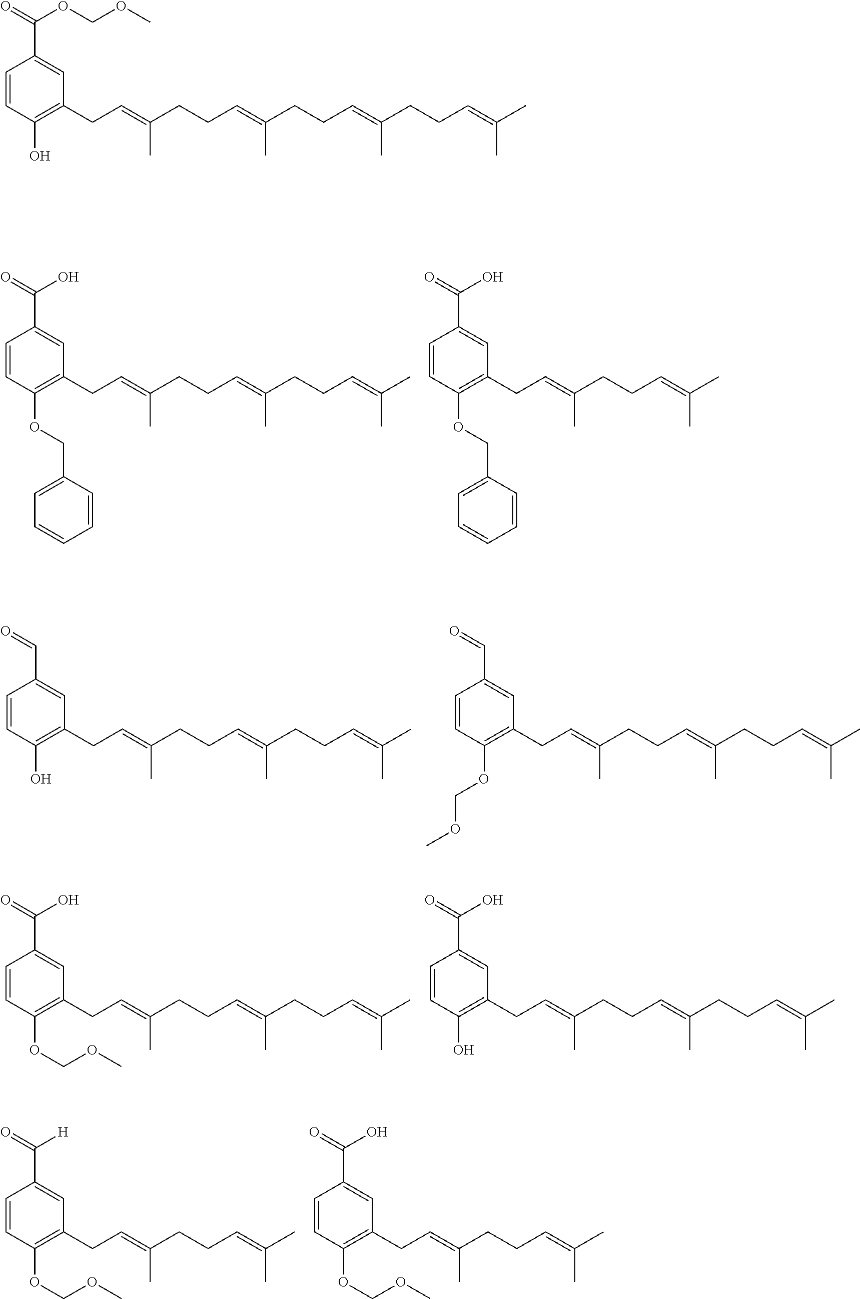

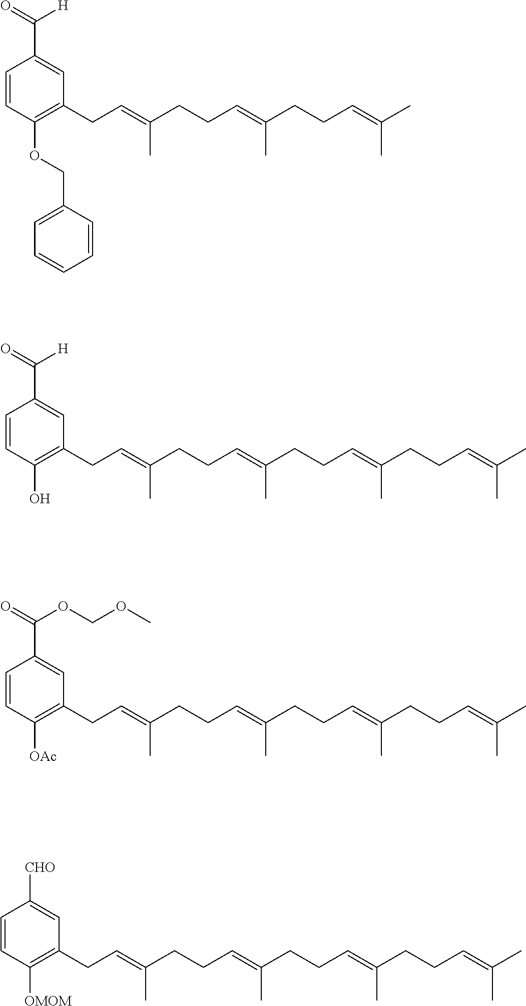

and salts, preferably pharmaceutically acceptable salts, solvates and prodrugs thereof.

Unless otherwise stated, the compounds of the invention are also meant to include compounds which differ only in the presence of one or more isotopically enriched atoms. For example, compounds having the present structures except for the replacement of a hydrogen by a deuterium or tritium, or the replacement of a carbon by a ¹³C— or ¹⁴C-enriched carbon or ¹⁵N-enriched nitrogen are within the scope of this invention.

The term “pharmaceutically acceptable salts, solvates and prodrugs thereof” refers to salts, solvates, or prodrugs which, upon administration to the recipient are capable of providing (directly or indirectly) a compound as described herein. However, it will be appreciated that non-pharmaceutically acceptable salts also fall within the scope of the invention since those may be useful in the preparation of pharmaceutically acceptable salts. The preparation of salts, prodrugs and derivatives can be carried out by methods known in the art. Preferably, “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

For instance, pharmaceutically acceptable salts of compounds provided herein are synthesized from the parent compound which contains a basic or acidic moiety by conventional chemical methods. Generally, such salts are, for example, prepared by reacting the free acid or base forms of these compounds with a stoichiometric amount of the appropriate base or acid in water or in an organic solvent or in a mixture of the two. Generally, nonaqueous media like ether, ethyl acetate, ethanol, isopropanol or acetonitrile are preferred. Examples of the acid addition salts include mineral acid addition salts such as, for example, hydrochloride, hydrobromide, hydroiodide, sulphate, nitrate, phosphate, and organic acid addition salts such as, for example, acetate, maleate, fumarate, citrate, oxalate, succinate, tartrate, malate, mandelate, methanesulphonate and p-toluenesulphonate. Examples of the alkali addition salts include inorganic salts such as, for example, sodium, potassium, calcium, ammonium, magnesium, aluminium and lithium salts, and organic alkali salts such as, for example, ethylenediamine, ethanolamine, N,N-dialkylenethanolamine, triethanolamine, glucamine and basic aminoacids salts.

The term “prodrug” as used in this application is defined here as meaning a chemical compound having undergone a chemical derivation such as substitution or addition of a further chemical group to change (for pharmaceutical use) any of its physico-chemical properties, such as solubility or bioavailability, e.g. ester and ether derivatives of an active compound that yield the active compound per se after administration to a subject. Examples of well known methods of producing a prodrug of a given acting compound are known to those skilled in the art and can be found e.g. in Krogsgaard-Larsen et al., Textbook of Drug Design and Discovery, Taylor & Francis (April 2002). The term “solvate” according to this invention is to be understood as meaning any form of the compound of the invention which has another molecule (most likely a polar solvent) attached to it via non-covalent bonding. Examples of solvates include hydrates and alcoholates, e.g. methanolate.

Particularly favoured prodrugs are those that increase the bioavailability of the compounds of this invention when such compounds are administered to a patient (e.g., by allowing an orally administered compound to be more readily absorbed into the blood) or which enhance delivery of the parent compound to a biological compartment (e.g., the brain or lymphatic system) relative to the parent species.

The preparation of salts, solvates and prodrugs can be carried out by methods known in the art. It will be appreciated that non-pharmaceutically acceptable salts, solvates or prodrugs also fall within the scope of the invention since those may be useful in the preparation of pharmaceutically acceptable salts, solvates or prodrugs.

The compounds of the invention may be in crystalline form either as free compounds or as solvates (e.g. hydrates) and it is intended that both forms are within the scope of the present invention. Methods of solvation are generally known within the art. Suitable solvates are pharmaceutically acceptable solvates. In a particular embodiment the solvate is a hydrate.

The compounds of formula (I) according to the present invention or their salts or solvates are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I), or of its salts, solvates or prodrugs.

The compounds of the present invention represented by the above described formula (I) may include enantiomers depending on the presence of chiral centres or isomers depending on the presence of multiple bonds (e.g. Z, E). The single isomers, enantiomers or diastereoisomers and mixtures thereof fall within the scope of the present invention.

Another aspect of the present invention is a compound of formula (I) as defined above, for use as a medicament.

The present invention further provides pharmaceutical compositions comprising at least a novel compound of formula (I) of the present invention, or pharmaceutically acceptable salts, solvates or prodrugs thereof and at least one pharmaceutically acceptable carrier, adjuvant, and/or vehicle, for administration to a patient.

The term “carrier, adjuvant and/or vehicle” refers to a molecular entities or substances with which the active ingredient is administered. Such pharmaceutical carriers, adjuvants or vehicles can be sterile liquids, such as water and oils, including those of petroleum, animal, vegetable or synthetic origin, such as peanut oil, soybean oil, mineral oil, sesame oil and the like, excipients, disgregants, wetting agents or diluents. Suitable pharmaceutical carriers are described in “Remington's Pharmaceutical Sciences” by E. W. Martin.

Examples of pharmaceutical compositions include any solid (tablets, pills, capsules, granules etc.) or liquid (solutions, suspensions or emulsions) composition for oral, topical or parenteral administration.

In a preferred embodiment the pharmaceutical compositions are in oral form. Suitable dosage forms for oral administration may be tablets or capsules and may contain conventional excipients known in the art such as binding agents, for example syrup, acacia, gelatin, sorbitol, tragacanth, or polyvinylpyrrolidone; fillers, for example lactose, sugar, maize starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulfate.

The solid oral compositions may be prepared by conventional methods of blending, filling or tabletting. Repeated blending operations may be used to distribute the active agent throughout those compositions employing large quantities of fillers. Such operations are conventional in the art. The tablets may for example be prepared by wet or dry granulation and optionally coated according to methods well known in normal pharmaceutical practice, in particular with an enteric coating.

The pharmaceutical compositions may also be adapted for parenteral administration, such as sterile solutions, suspensions or lyophilized products in the appropriate unit dosage form. Adequate excipients can be used, such as bulking agents, buffering agents or surfactants.

The mentioned formulations will be prepared using standard methods such as those described or referred to in the Spanish and US Pharmacopoeias and similar reference texts.

Administration of the novel compounds of formula (I) or compositions of the present invention may be by any suitable method, such as intravenous infusion, oral preparations, and intraperitoneal and intravenous administration. Oral administration is preferred because of the convenience for the patient and the chronic character of many of the diseases to be treated.

The novel compounds and compositions of this invention may be used with other drugs to provide a combination therapy. The other drugs may form part of the same composition, or be provided as a separate composition for administration at the same time or at different time.

Another aspect of the present invention is the use of a compound of formula (I*)

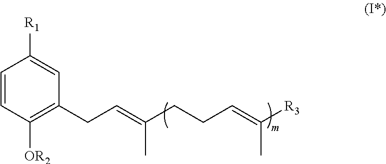

wherein
m is an integer selected from 0, 1, 2, 3, 4, and 5;
R₁ is selected from C₁-C₁₂ alkoxy, —CH₂—O—CH₃, —OH, —C(═O)OR₄, —CHO and —CONH—R₅,
wherein R₄ is selected from hydrogen, C₁-C₆ alkyl, —CH₂-Ph, —CH₂—O—CH₃,
and R₅ is C₁-C₆ alkyl,
R₂ is selected from hydrogen, phenyl, benzyl, —COR_{E}, C₁-C₆ alkyl and —CH₂—O—CH₃,
wherein R₆ is selected from hydrogen and C₁-C₆ alkyl,
R₃ is selected from —CH₃ and

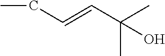

and salts, preferably any pharmaceutically acceptable salts, solvates and prodrugs thereof;
in the manufacture of a medicament for the treatment and/or profilaxis of a cognitive, neurodegenerative or neuronal disease or disorder.

A further aspect is a compound of formula (I*) for use in the treatment and/or profilaxis of a cognitive, neurodegenerative or neuronal disease or disorder.

For this use, preferred compounds of formula (I*) are the following:

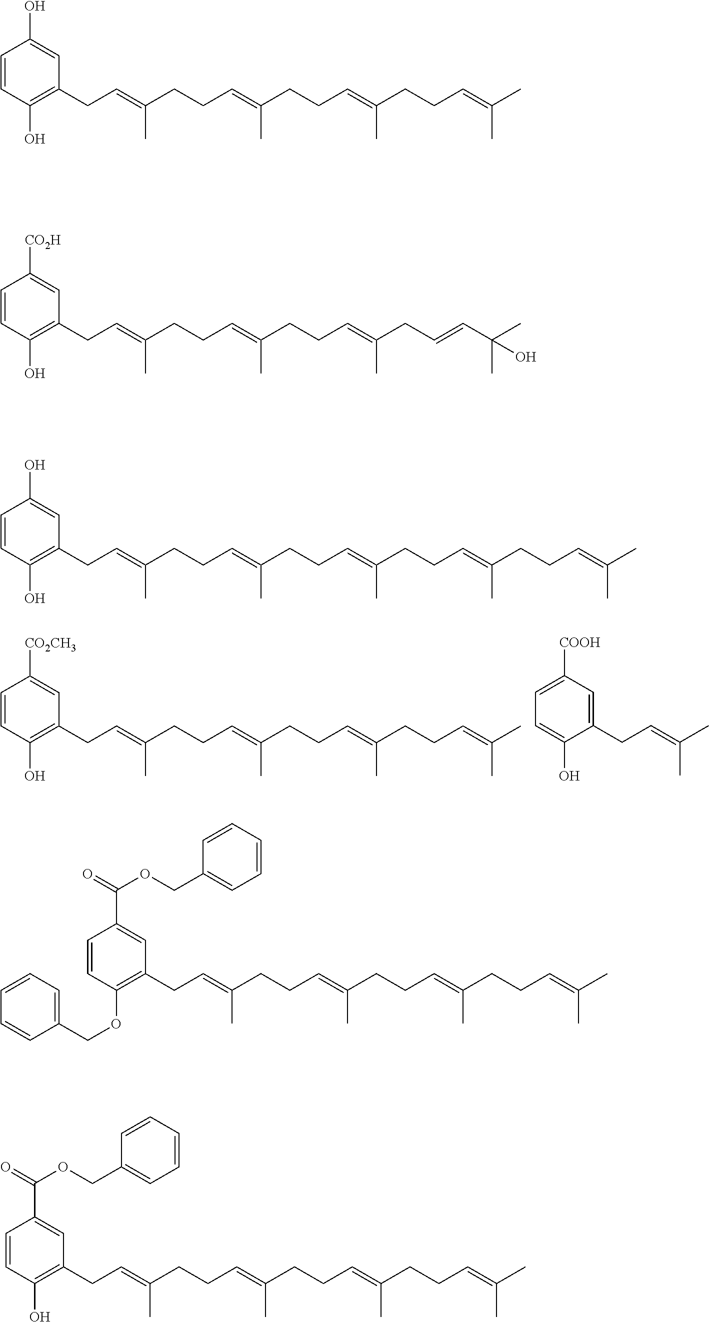

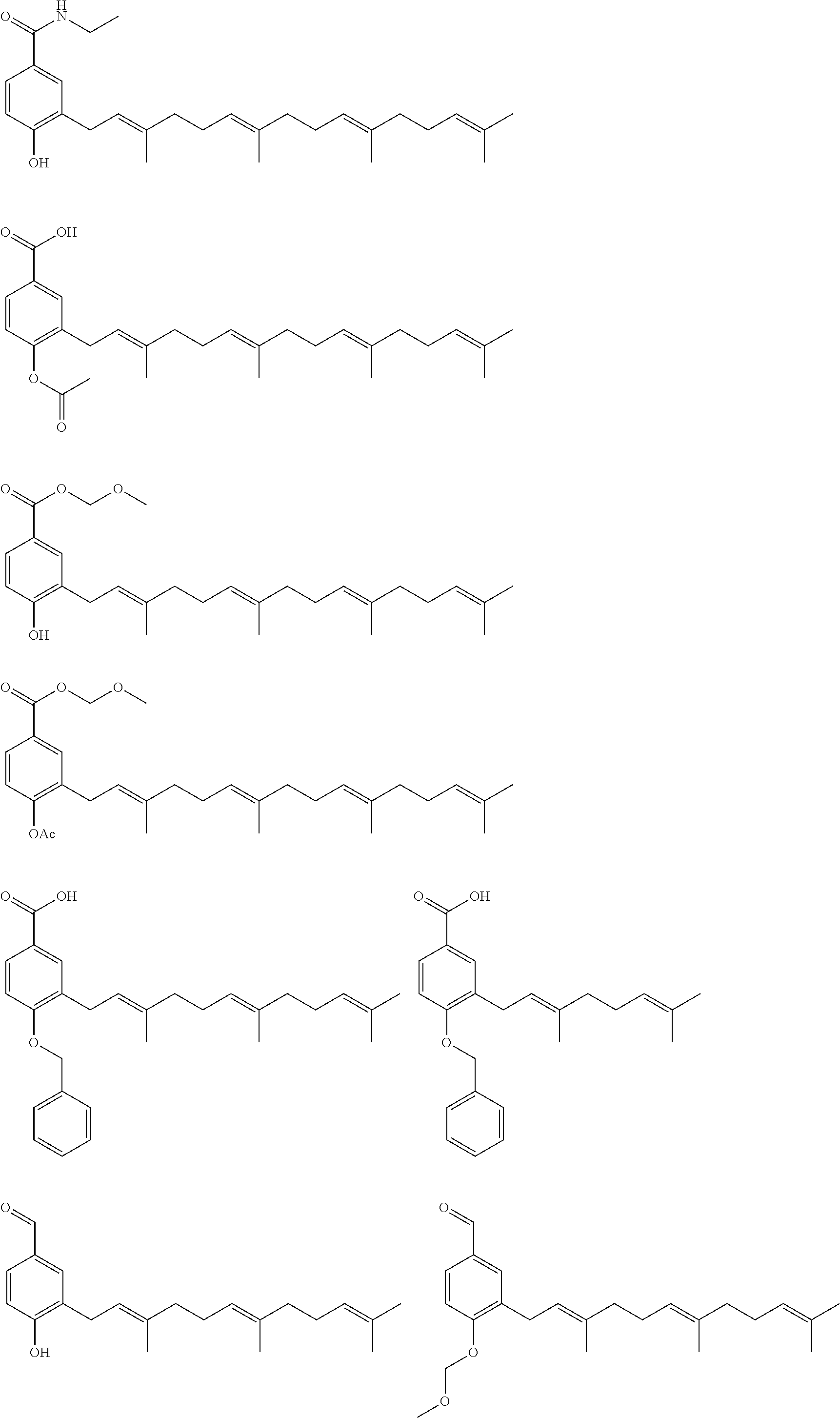

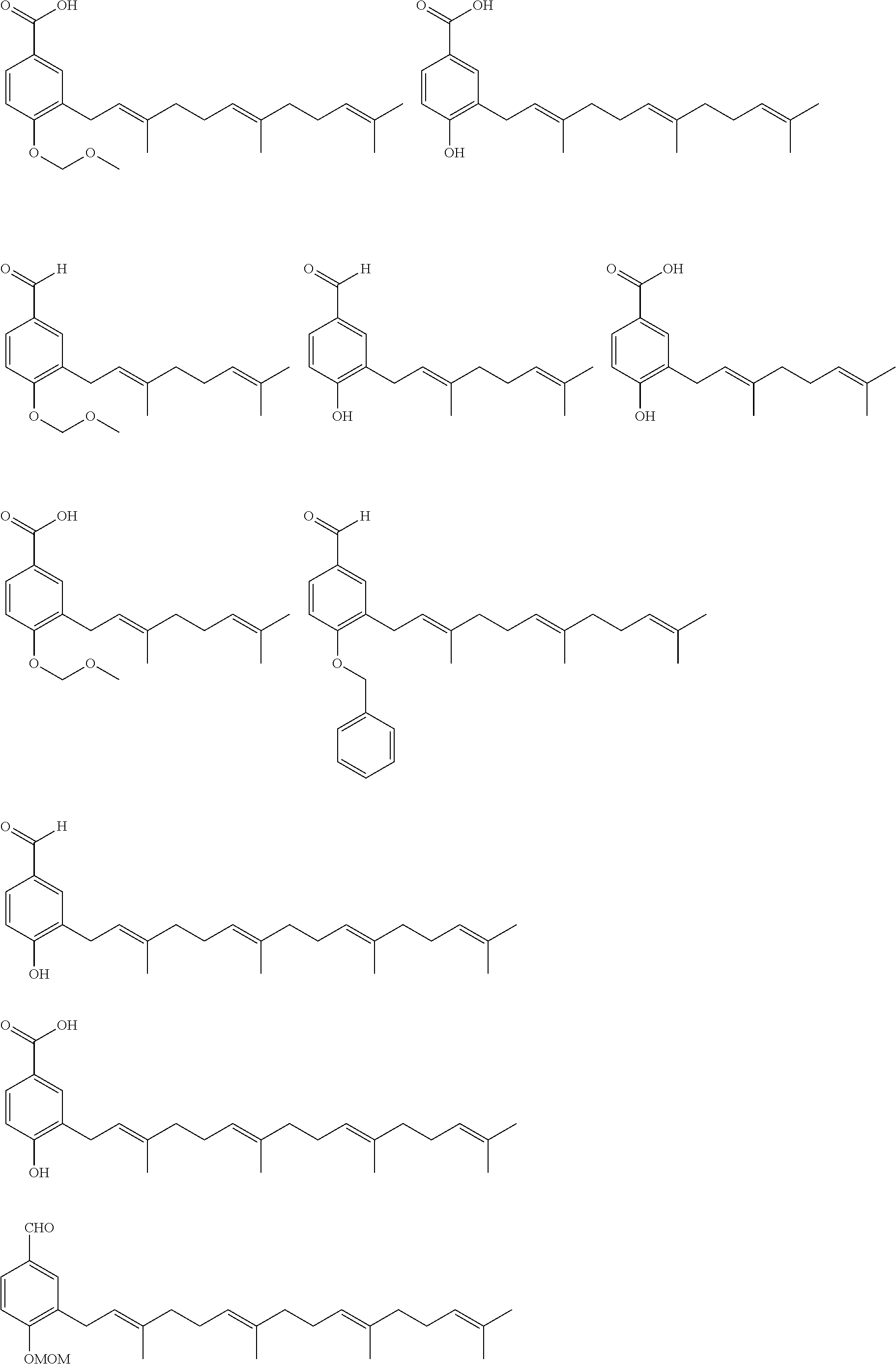

Within the frame of the present invention, “a cognitive, neurodegenerative or neuronal disease or disorder” refers to any disease, disorder or condition selected from, but not limited to, chronic neurodegenerative conditions including dementias such as Alzheimer's disease, Parkinson's disease, progressive supranuclear palsy, subacute sclerosing panencephalitic parkinsonism, postencephalitic parkinsonism, pugilistic encephalitis, guam parkinsonism-dementia complex, Pick's disease, corticobasal degeneration, frontotemporal dementia, Huntington's Disease, AIDS associated dementia, amyotrophic lateral sclerosis, multiple sclerosis and neurotraumatic diseases such as acute stroke, epilepsy, mood disorders such as depression, schizophrenia and bipolar disorders, promotion of functional recovery post stroke, cerebral bleeding, such as cerebral bleeding due to solitary cerebral amyloid angiopathy, mild cognitive impairment, Hereditary Cerebral Hemorrhage with Amyloidosis of the Dutch-Type, cerebral Amyloid angiopathy, ischaemia, brain injury, especially traumatic brain injury, Down's syndrome, Lewy body disease, inflammation and chronic inflammatory diseases

Preferred diseases or disorders are diabetes, chronic neurodegenerative conditions including dementias such as Alzheimer's disease and Parkinson's disease, Huntington's Disease, amyotrophic lateral sclerosis, multiple sclerosis and neurotraumatic diseases such as acute stroke, epilepsy, mood disorders such as depression, schizophrenia and bipolar disorders, promotion of functional recovery post stroke, cerebral bleeding, mild cognitive impairment, ischaemia, brain injury, especially traumatic brain injury, inflammation and chronic inflammatory diseases.

Especially preferred diseases are Alzheimer's Disease, Parkinson's Disease, multiple sclerosis, stroke, epilepsy, mood disorders, ischaemia, brain injury and chronic inflammatory diseases.

Another aspect of the present invention is a method of treating and/or preventing a cognitive, neurodegenerative or neuronal disease or disorder, which method comprises administering to a patient in need of such a treatment a therapeutically effective amount of at least one compound of formula (I*) as defined above or a pharmaceutical composition thereof.

The term “cognitive, neurodegenerative or neuronal disease or disorder” shall be interpreted as indicated above.

The disease or disorder is preferably selected from, but not limited to, chronic neurodegenerative conditions including dementias such as Alzheimer's disease, Parkinson's disease, progressive supranuclear palsy, subacute sclerosing panencephalitic parkinsonism, postencephalitic parkinsonism, pugilistic encephalitis, guam parkinsonism-dementia complex, Pick's disease, corticobasal degeneration, frontotemporal dementia, Huntington's Disease, AIDS associated dementia, amyotrophic lateral sclerosis, multiple sclerosis and neurotraumatic diseases such as acute stroke, epilepsy, mood disorders such as depression, schizophrenia and bipolar disorders, promotion of functional recovery post stroke, cerebral bleeding, such as cerebral bleeding, due to solitary cerebral amyloid angiopathy, mild cognitive impairment, Hereditary Cerebral Hemorrhage with Amyloidosis of the Dutch-Type, cerebral Amyloid angiopathy, ischaemia, brain injury, especially traumatic brain injury, Down's syndrome, Lewy body disease, inflammation and chronic inflammatory diseases.

Generally a “therapeutically effective amount” of the compound of the invention or a pharmaceutical composition thereof will depend on the relative efficacy of the compound chosen, the severity of the disorder being treated and the weight of the sufferer. However, active compounds will typically be administered once or more times a day for example 1, 2, 3 or 4 times daily, with typical total daily doses in the range of from 0.1 to 1000 mg/kg/day.

The term “treatment” or “to treat” in the context of this specification means administration of a compound or formulation according to the invention to prevent, ameliorate or eliminate the disease or one or more symptoms associated with said disease. “Treatment” also encompasses preventing, ameliorating or eliminating the physiological sequelae of the disease.

The term “ameliorate” in the context of this invention is understood as meaning any improvement on the situation of the patient treated—either subjectively (feeling of or on the patient) or objectively (measured parameters).

In the following, the present invention is further illustrated by examples. They should in no case be interpreted as a limitation of the scope of the invention as defined in the claims.

### EXAMPLES

### Example 1

### Description of the Sponge and the Collection Site

Sarcotragus was collected in May 2001 by hand using scuba diving near Colomer Island, (Formentor, Mallorca Island, Spain 39° 56′ 617″ N y 3° 07′ 860″ E) in a cave at 42 m depth. A voucher specimen (ORMA000312) is deposited at PharmaMar.

### Example 2

### Extraction and Isolation of Compounds

The frozen sponge (488 g) was diced and extracted with isopropanol (3×1000 ml) at room temperature. The combined extracts were concentrated under reduced pressure to yield a crude of 16.09 g. This material was subjected to VLC on Lichroprep RP-18 with a stepped gradient from H₂O to MeOH and subsequently MeOH/CH₂Cl₂ (1:1). Fractions eluted with 100% of MeOH were chromatographed on Silica gel with a stepped gradient from Hexane/Ethyl Acetate and subsequently MeOH/EtOAc (1:1). Fractions eluted with Hexane/EtOAc (7:3) were subjected to semipreparative reversed phase HPLC separation (SymmetryPrep C-18, 19×300 mm, gradient H₂O—AcN+0.1% formic acid from 80 to 100% AcN in 10 min followed by 100% of AcN in 30 min, UV detection at 254 and 290 nm), to afford the pure compounds Compound 2, Compound 4 and Compound 5. Fractions eluted with Hexane/EtOAc (1:1) afforded the pure compound Compound 1. Fractions eluted with MeOH/EtOAc (1:1) were subjected to semipreparative reversed phase HPLC separation (SymmetryPrep C-18, 19×300 mm, gradient H₂O—AcN+0.1% formic acid from 60 to 100% AcN in 45 min, UV detection at 254 and 290 nm) giving the pure compound Compound 3.

### Compound 3:

¹H NMR (400 MHz, CDCl₃) δ 7.9 (br.s), 6.9 (br.s), 5.6 (m), 5.2 (br.s), 5.1 (m), 2.6 (m), 2.2-2.0 (m), 1.8 (s), 1.6 (s), 1.58 (s), 1.3 (s).

¹³C NMR (100 MHz, CDCl₃) δ. 170.1, 159.2, 138.9, 139.0, 135.5, 133.5, 132.5, 130.4, 126.9, 125.6, 125.2, 123.6, 121.8, 121.1, 115.6, 71.0, 42.3, 39.6, 29.7, 26.7, 26.1, 16.2, 1.60.

Compounds 1 to 5 are as follows:

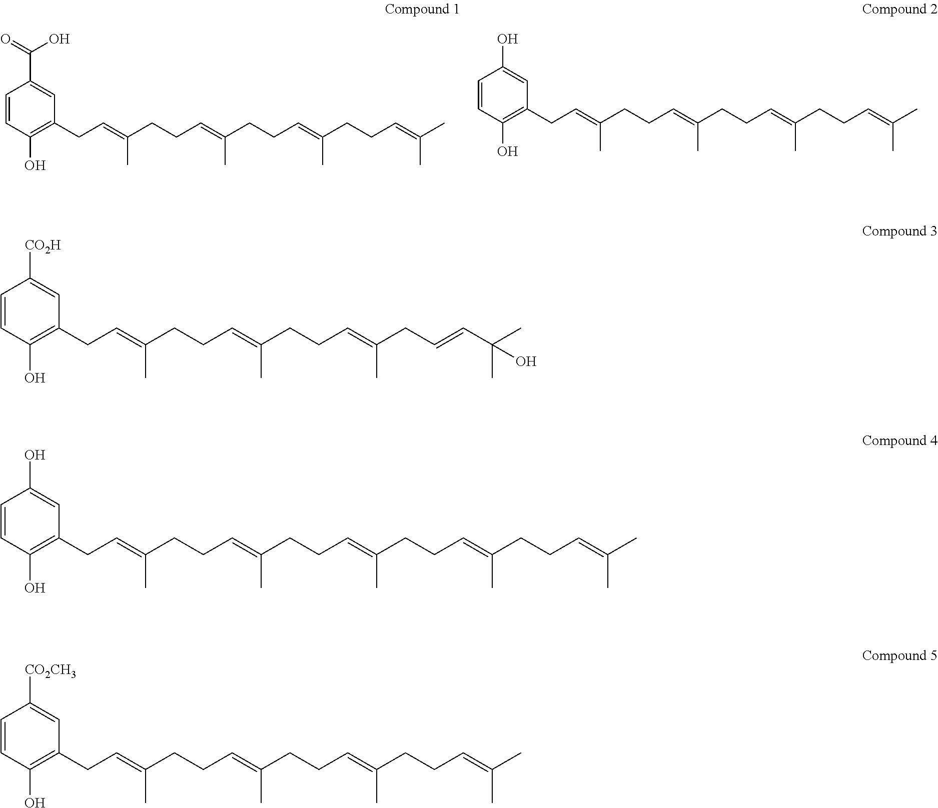

### Preparation

Following the above-indicated general reaction scheme, the following compounds were obtained:

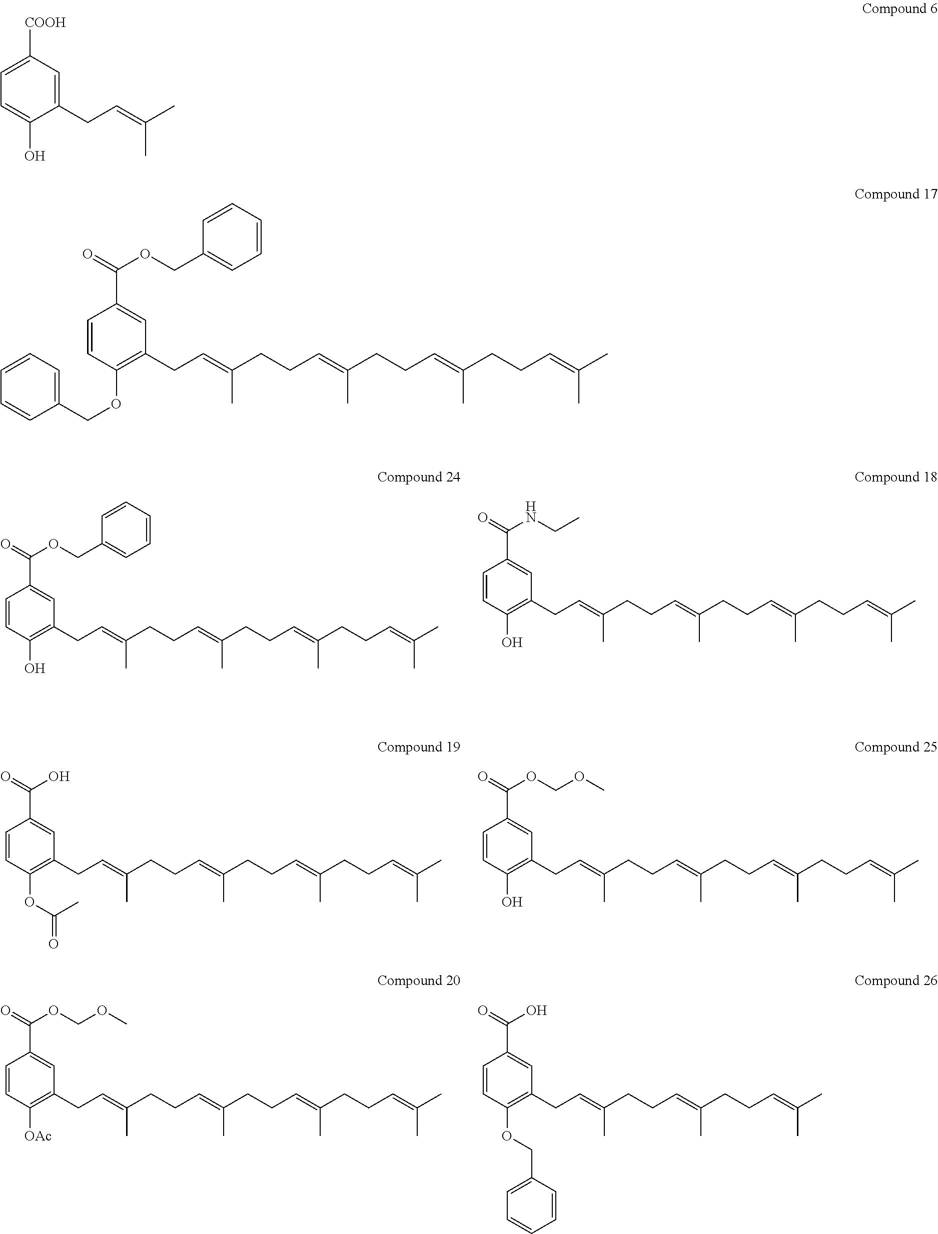

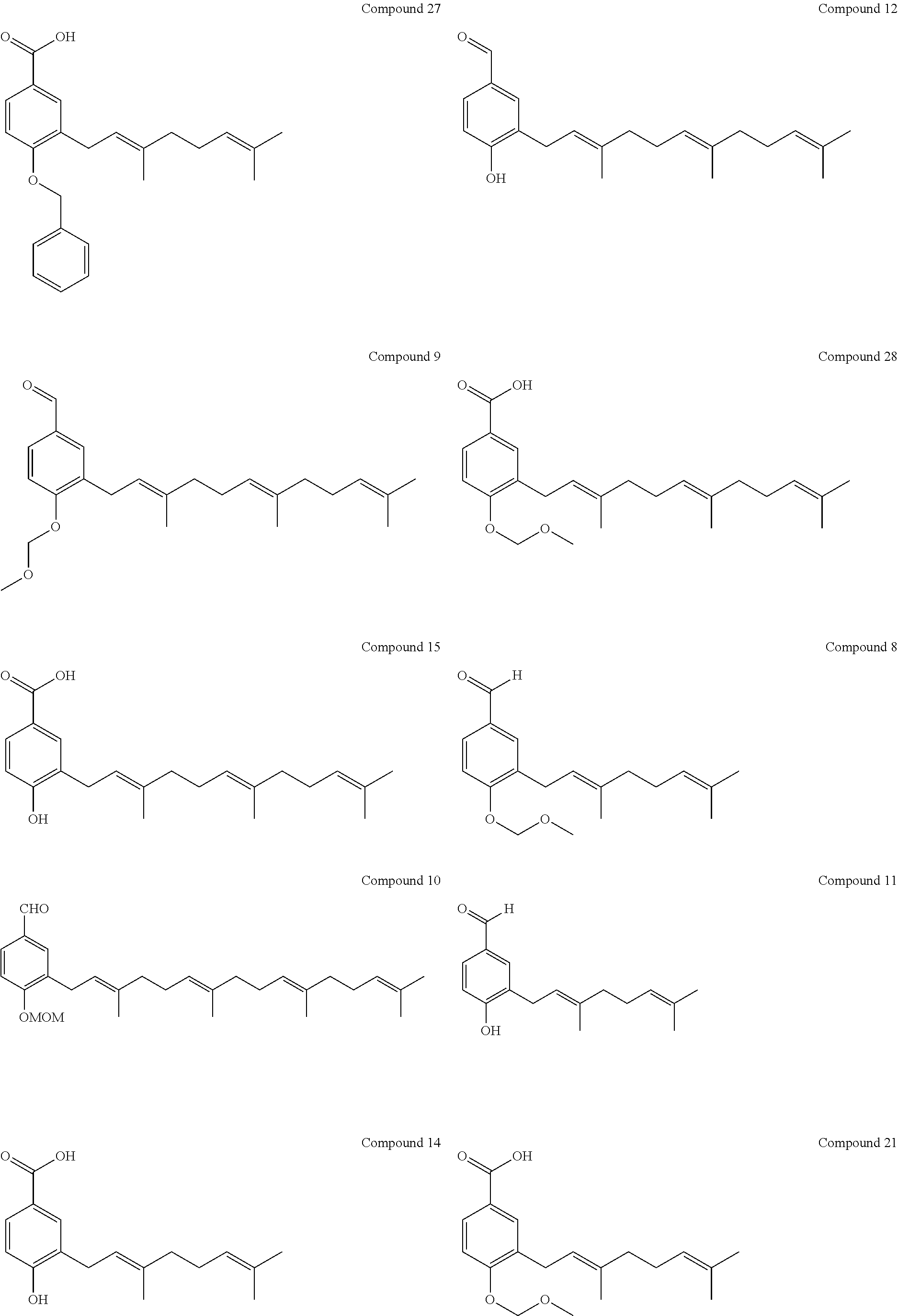

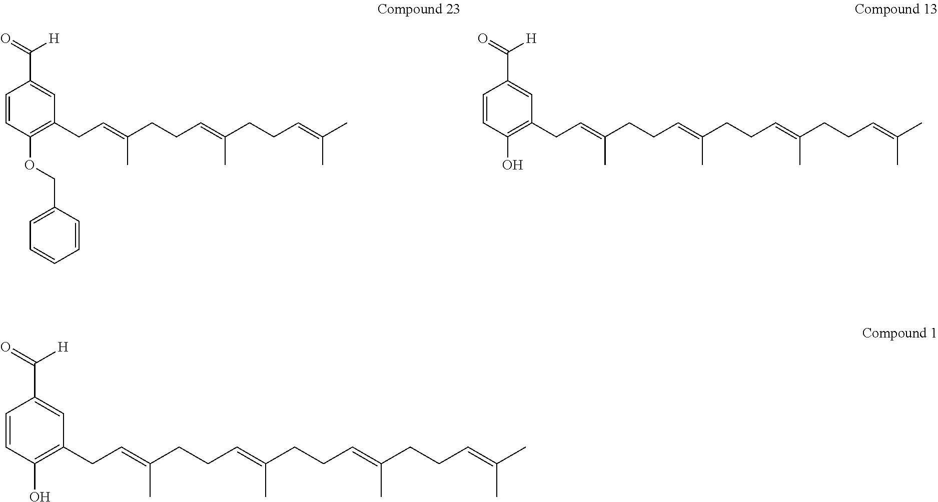

The detailed preparation of some of the compounds is described hereinafter:

### Example 3

### Preparation of the Compounds of Formula (I)

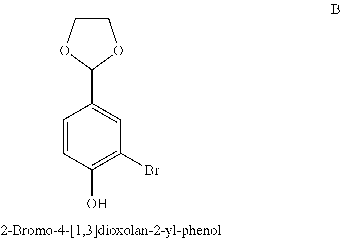

To a solution of 3-bromo-4-hydroxybenzaldehyde (5.0 g, 24.8 mmoles) in anhydrous toluene (75 mL), Ethylene glycol (1.66 mL, 29.8 mmoles) and p-Toluenesulfonic acid monohydrate (473 mg, 2.49 mmoles) are added. The resulting mixture is heated to 135° C., preferably using a Dean-Stark, during 5 hours; once this time has elapsed, the mixture is brought to room temperature. Triethylamine (2 mL) is added, and the solvent is eliminated under reduced pressure. A purification using a silica gel chromatographic column is performed, using as the eluent a mixture of Ethyl acetate/Hexane in a relation of 1:2, obtaining 5.4 g of a white, solid product (Yield: 90%).

¹H NMR (400 MHz, CD₃OD) δ 7.54 (d, 1H, J=2.0), 7.24 (dd, 1H, J=2.0, 8.3), 6.88 (m, 1H), 5.63 (s, 1H), 4.02 (m, 5H)

¹³C NMR (100 MHz, CD₃OD) δ 156.27, 132.59, 132.01, 128.28, 116.87, 110.52, 104.18, 66.30.

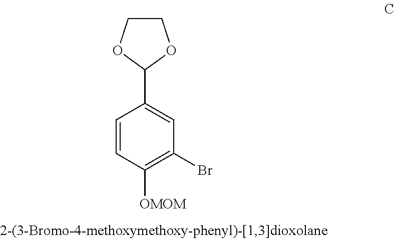

To a solution of 2-Bromo-4-[1,3]dioxolan-2-yl-phenol (5.3 g, 22.0 mmoles) in anhydrous THF (75 mL), cooled to 0° C. and under nitrogen atmosphere, DIPEA (9.42 mL, 54.0 mmoles) is slowly added. The resulting mixture is stirred during 15 minutes at 0° C. Once this time has elapsed, CIMOM (3.48 mL, 43.0 mmoles) is added dropwise, and the reaction is stirred during 16 hours at room temperature. The mixture is dried under reduced pressure, and a purification using a silica gel column is performed, using as the mobile phase a mixture of Ethyl acetate/Hexane in a relation of 1:10, obtaining 6.0 g of a transparent, liquid product (Yield: 95%).

¹H NMR (400 MHz, CDCl₃) [symbol] ppm 7.66 (s, 1H), 7.33 (dd, 1H, J=1.5, 8.4), 7.12 (d, 1H, J=8.5), 5.72 (s, 1H), 5.20 (m, 2H), 4.04 (m, 4H), 3.48 (m, 3H).

¹³C NMR (100 MHz, CDCl₃) [symbol] ppm 154.32, 132.92, 131.51, 126.78, 115.65, 112.73, 102.68, 95.00, 65.27, 56.34.

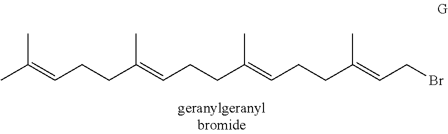

To a solution of geranyllinalool (5 g; 170 mmoles) in anhydrous diethyl ether (20 mL), a solution of phosphorus tribromide (0.81 mL; 8.61 mmoles) is added dropwise, at 0° C., under nitrogen atmosphere. The mixture is stirred during 3 hours at said temperature, and subsequently the mixture is diluted with another 20 mL of diethyl ether. The reaction is stopped adding a saturated solution of NaHCO₃ (20 mL), observing bubbles. 20 mL water are added. An extraction using diethyl ether (2×50 mL) is performed, the ether phase is dried with anhydrous Na₂SO₄, filtered, and dried under reduced pressure. A purification in silica gel column is performed, using as mobile phase a mixture of AcOEt/Hexanol 1:20, obtaining a yellow, oily product.

¹H NMR (400 MHz, CDCl₃) [symbol] ppm 5.53 (dt, J=8.44, 1.17 Hz, 1H), 5.17-5.03 (m, 3H), 4.02 (d, J=8.43 Hz, 2H), 2.19-1.92 (m, 12H), 1.73 (d, J=1.23 Hz, 3H), 1.68 (d, J=0.86 Hz, 3H), 1.60 (s, 9H).

¹³C NMR (100 MHz, CDCl₃) [symbol] ppm 143.60, 135.63, 134.95, 131.25, 124.36, 124.16, 123.37, 120.52, 39.72, 39.66, 39.53, 29.67, 26.76, 26.59, 26.10, 25.69, 17.68, 16.05, 16.00, 15.97.

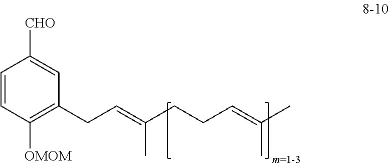

### General Procedure for the Synthesis of Products 8-10:

To a solution of 2-(3-Bromo-4-methoxymethoxy-phenyl)-[1,3]dioxolane (C) (3.46 mmoles) in anhydrous toluene (6 mL) and anhydrous diethyl ether (10 mL), a small quantity of molecular sieves are added. To said solution, at room temperature and under nitrogen atmosphere, n-BuLi (1.3 equivalents, 4.50 mmoles; 1.6M solution in hexane) is added, stirring the mixture during 5 minutes. Subsequently, CuBr.DMS (0.6 equivalents, 2.07 mmoles) is added, Stirring the mixture during another 30 minutes; once the time has elapsed, the corresponding prenyl bromide (1.1 equivalents, 3.80 mmoles) is added. After 4 hours, the reaction is stopped adding an aqueous saturated solution of ammonium chloride (NH₄Cl) (5 mL); the resulting mixture is extracted with diethyl ether (2×50 mL) and the organic phase is washed with a 1N solution of Hydrochloric acid (HCl) (2×50 mL). The organic phase is dried over Sodium sulfate, filtered and dried under reduced pressure. Purification using a silica gel column is performed, using as the mobile phase a mixture of Ethyl acetate/Hexane in a 1:10 relation, obtaining the product as a transparent oil.

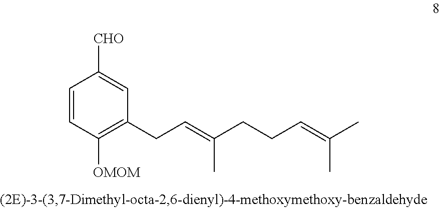

¹H NMR (400 MHz, CDCl₃ [symbol] ppm) 9.87 (s, 1H), 7.69 (m, 2H), 7.17 (d, J=9.09 Hz, 1H), 5.35-5.29 (m, 1H), 5.29 (s, 2H), 5.10 (dtdd, J=5.80, 4.34, 2.96, 1.45 Hz, 1H), 3.49 (s, 3H), 3.39 (d, J=7.41 Hz, 2H), 2.16-2.00 (m, 4H), 1.72 (d, J=1.04 Hz, 1H), 1.67 (d, J=1.09 Hz, 3H), 1.59 (s, 3H).

¹³C NMR (100 MHz, CDCl₃ [symbol] ppm) 191.22, 159.93, 137.05, 131.56, 131.52, 130.82, 130.43, 130.05, 124.10, 121.24, 113.20, 94.01, 56.28, 39.74, 28.43, 26.60, 25.68, 17.68, 16.14.

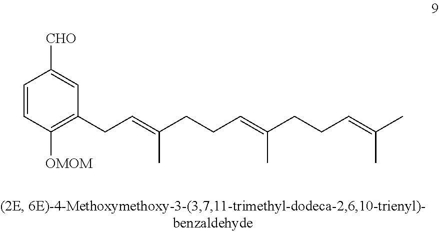

¹H-NMR (400 MHz, CDCl₃ [symbol] ppm) 9.87 (s; 1H); 7.70 (m; 2H); 7.17 (d; 1H; J=8.7 Hz); 5.33 (t; 1H; J=7.2 Hz); 5.29 (s; 3H); 5.10 (m; 2H); 3.49 (s; 3H); 3.39 (d; 2H; J=7.3 Hz); 2.05 (m; 8H); 1.73 (s; 3H) 1.67 (s; 3H) 1.59 (s; 6H)

¹³C-NMR (100 MHz, CDCl₃ [symbol] ppm) 191.21; 159.92; 137.08; 135.13; 131.54; 131.26; 130.83; 130.44; 130.05; 124.32; 123.99; 121.23; 113.21; 94.00; 56.27; 39.76; 39.69; 28.45; 26.72; 26.56; 25.67; 17.66; 16.17; 16.00.

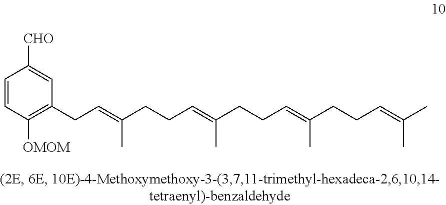

¹H NMR (400 MHz, CDCl₃δ ppm) 9.87 (s, 1H), 7.72-7.65 (m, 2H), 7.17 (d, J=9.02 Hz, 1H), 5.35-5.30 (m, 1H), 5.29 (s, 2H), 5.16-5.05 (m, 3H), 3.49 (s, 3H), 3.39 (d, J=7.30 Hz, 2H), 2.21-1.90 (m, 12H), 1.73 (s, 3H), 1.68 (s, 3H), 1.59 (s, 6H), 1.58 (s, 3H).

¹³C NMR (100 MHz, CDCl₃δ ppm) 191.17, 159.91, 137.07, 135.14, 134.88, 131.53, 131.21, 130.82, 130.43, 130.03, 124.37, 124.18, 123.99, 121.21, 113.20, 93.98, 56.25, 39.77, 39.69, 28.45, 26.74, 26.61, 25.67, 17.65, 16.17, 16.00, 15.97.

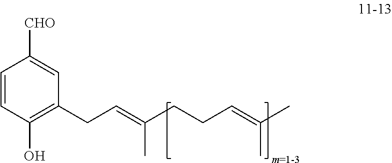

### General Procedure for the Synthesis of Products 11-13:

To a solution of the products 8-10 (024 mmoles), dissolved in methanol (10 mL), (+/−)-camphor-10-sulfonic acid (0.26 mmoles) is added. The resulting solution is heated to 70° C. during 4 hours. The reaction is stopped adding a saturated aqueous solution of NaHCO₃ (Sodium bicarbonate)(5 ml). It is extracted using diethyl ether (2×25 ml), and washed with water (1×25 ml) and brine (1×25 ml). The organic phase is dried with Sodium sulfate, filtered and dried under reduced pressure. A purification with a silica gel column is performed, using as the mobile phase a mixture of Ethyl acetate/Hexane in a relation of 1:4, obtaining a transparent, oily product.

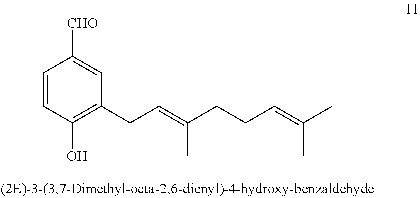

¹H NMR (400 MHz, CDCl₃δ ppm) 9.85 (s, 1H), 7.71-7.64 (m, 2H), 6.93 (d, J=8.77 Hz, 1H), 5.33 (dt, J=7.21, 1.28 Hz, 1H), 5.10-5.03 (m, 1H), 3.43 (d, J=7.20 Hz, 2H), 2.21-2.05 (m, 4H), 1.78 (d, J=0.66 Hz, 3H), 1.68 (d, J=0.86 Hz, 3H), 1.60 (d, J=0.50 Hz, 3H)

¹³C NMR (100 MHz, CDCl₃δ ppm) 191.15, 160.28, 139.83, 132.14, 131.96, 130.50, 129.99, 127.56, 123.62, 120.52, 116.28, 39.66, 29.57, 26.32, 25.67, 17.71, 16.27

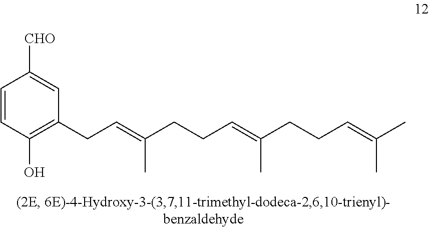

¹H NMR (400 MHz, CDCl₃δ ppm) 9.85 (s, 1H); 7.67 (m, 2H); 6.91 (d, 1H, J=8.8 Hz); 5.78 (s, 1H); 5.33 (t, 1H, J=7.4 Hz); 5.08 (m, 2H); 3.43 (d, 2H, J=7.1 Hz); 2.07 (m, 8H); 1.79 (s, 3H); 1.67 (s, 3H); 1.59 (s, 6H)

¹³C NMR (100 MHz, CDCl₃δ ppm) 191.0; 160.1; 139.9; 135.7; 131.9; 131.3; 130.4; 130.0; 127.4; 124.3; 123.4; 121.1; 120.4; 116.3; 39.6; 29.6; 26.6; 26.2; 25.6; 17.6; 16.3; 16.0.

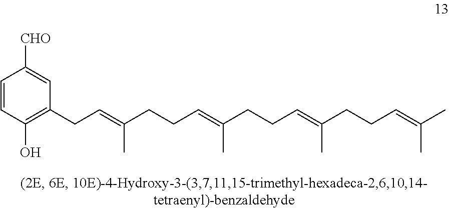

¹H NMR (400 MHz, CDCl₃δ ppm) 9.84 (s, 1H), 7.67 (m, 2H), 6.92 (d, J=8.34 Hz, 1H), 5.33 (t, J=6.59 Hz, 1H), 5.20-5.02 (m, 3H), 3.43 (d, J=6.70 Hz, 2H), 2.30-1.87 (m, 12H), 1.79 (s, 3H), 1.67 (s, 3H), 1.60 (s, 9H).

¹³C NMR (100 MHz, CDCl₃δ ppm) 191.21, 139.71, 135.70, 134.94, 131.99, 131.25, 130.47, 129.90, 127.64, 124.37, 124.17, 123.50, 121.21, 120.51, 116.23, 39.69, 39.64, 29.48, 26.75, 26.56, 26.33, 26.27, 25.68, 17.67, 16.31, 16.06, 15.99.

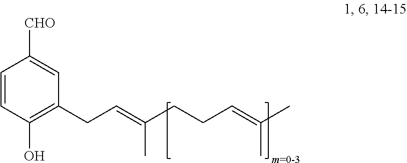

### General Procedure for the Synthesis of Products 1, 6, 14-15:

To a solution of 4-hydroxy-3-(3-methyl-but-2-enyl)-benzaldehyde or of aldehyde 11-13 (0.43 mmoles) in a mixture of THF/H₂O (2.5 mL/0.5 mL) and 2-methyl-2-butene (0.1 mL), Sodium dihydrogen phosphate (1.01 mmoles) and sodium chlorite (1.06 mmoles) are added one after the other. The reaction is stirred during 4 hours at room temperature; once the time has elapsed, the mixture is neutralised using a 1N solution of Hydrochloric acid (HCl), until a slight acidification occurs (pH 4-5). Water (20 mL) is added, and it is extracted with CH₂Cl₂ (2×25 mL); the organic phase is dried with Sodium sulfate, filtered and dried under reduced pressure. A purification using a chromatographic column is performed, using as eluent a mixture of Dichloromethane with 3% of Methanol.

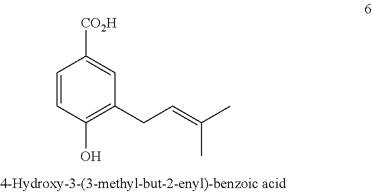

¹H NMR (400 MHz, CD₃OD [symbol] ) ppm 7.74 (d, J=1.74 Hz, 1H), 7.70 (dd, J=8.35, 1.99 Hz, 1H), 6.78 (d, J=8.36 Hz, 1H), 5.45-5.20 (m, 1H), 3.30 (d, J=3.44 Hz, 2H), 1.75 (d, J=0.84 Hz, 3H), 1.72 (s, 3H).

¹³C NMR (100 MHz, CD₃OD δ) ppm 170.69, 161.06, 133.72, 132.63, 130.48, 129.39, 123.41, 122.90, 115.37, 29.12, 26.03, 17.91.

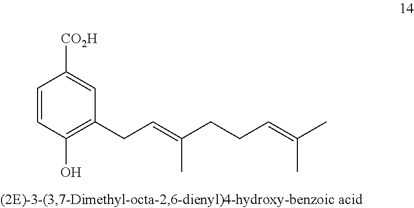

¹H NMR (400 MHz, CDCl₃δ ppm) 7.93-7.84 (m, 2H), 6.85 (d, J=8.88 Hz, 1H), 5.33 (dt, J=7.16, 1.17 Hz, 1H), 5.12-5.04 (m, 1H), 3.41 (d, J=7.17 Hz, 2H), 2.21-2.03 (m, 4H), 1.77 (d, J=0.74 Hz, 3H), 1.68 (d, J=0.83 Hz, 3H), 1.60 (d, J=0.52 Hz, 3H)

¹³C NMR (100 MHz, CDCl₃δ ppm) 172.20, 159.78, 139.38, 132.75, 132.23, 130.67, 127.29, 123.97, 121.74, 121.13, 115.88, 39.90, 29.67, 26.63, 25.87, 17.92, 16.46.

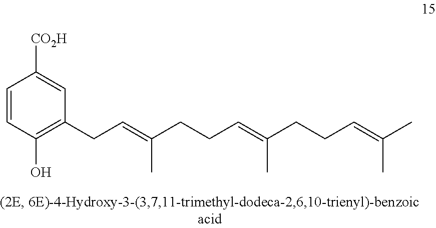

¹H NMR (400 MHz, CDCl₃ δ ppm) 7.98-7.84 (m, 2H), 6.85 (d, J=8.86 Hz, 1H), 5.33 (t, J=6.73 Hz, 1H), 5.19-5.04 (m, 2H), 3.42 (d, J=6.82 Hz, 2H), 2.05 (ddd, J=28.04, 13.03, 6.46 Hz, 8H), 1.79 (s, 3H), 1.67 (s, 3H), 1.60 (s, 6H)

¹³C NMR (100 MHz, CDCl₃□ ppm 171.81, 159.54, 139.57, 135.68, 132.57, 131.28, 130.52, 126.79, 124.33, 123.50, 121.61, 120.78, 115.78, 39.66, 29.71, 26.67, 26.32, 25.67, 17.67, 16.31, 16.04.

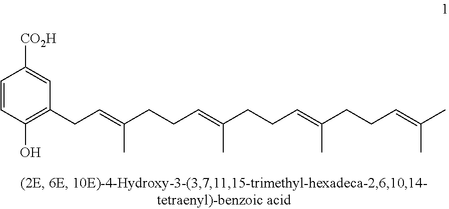

¹H NMR (400 MHz, CDCl₃ [symbol] ppm) 7.93-7.85 (m, 2H), 6.85 (d, J=6.95 Hz, 1H), 5.33 (t, J=6.98 Hz, 1H), 5.18-5.03 (m, 3H), 3.42 (d, J=6.67 Hz, 2H), 2.20-1.89 (m, 12H), 1.80 (s, 3H), 1.68 (s, 1H), 1.62-1.57 (m, 9H).

¹³C NMR (100 MHz, CDCl₃δ ppm 171.53, 159.53, 139.64, 135.72, 134.93, 132.59, 130.54, 126.76, 124.39, 124.21, 123.50, 121.61, 120.77, 115.80, 39.70, 39.65, 29.76, 26.76, 26.58, 26.35, 25.68, 17.67, 16.32, 16.06, 16.00.

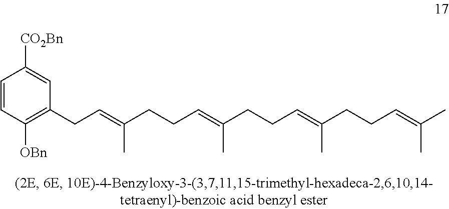

Benzyl bromide (43 mg, 0.25 mmol) was added portion wise to a suspension of (2E,6E, 10E)-4-Hydroxy-3-(3,7,11,15-tetramethyl-hexadeca-2,6,10,14-tetraenyl)-benzoic acid (100 mg, 0.25 mmol) and K₂CO₃ (34 mg, 0.25 mmol) in DMF (1.2 mL) and the mixture was stirred for four hours. After two hours the amber solution with K₂CO₃ in suspension turned to a colourless solution with a white suspension. Water was added and the mixture was extracted with Ethyl ether (25 mL). The ether phase was washed eight times with water (10 mL) and one time with brine, and the solvent evaporated. The purification was performed with radial chromatography 10:1 (Hexane/Ethyl acetate).

¹H NMR (400 MHz, CDCl₃δ ppm 7.94-7.90 (m, 2H), 7.47-7.30 (m, 10H), 6.91 (d, J=9.23 Hz, 1H), 5.34 (s, 2H), 5.34-5.30 (m, 1H), 5.15 (s, 2H), 5.14-5.07 (m, 3H), 3.41 (d, J=7.23 Hz, 2H), 2.16-1.92 (m, 12H), 1.69 (d, J=1.14 Hz, 3H), 1.67 (d, J=0.91 Hz, 3H), 1.60 (s, 3H), 1.59 (s, 6H).

¹³C NMR (100 MHz, CDCl₃δ ppm 166.37, 160.32, 136.71, 136.61, 136.45, 135.03, 134.86, 131.19, 130.55, 129.45, 128.56, 128.50, 128.03, 127.98, 127.16, 124.42, 124.27, 124.17, 122.40, 121.66, 110.81, 70.01, 66.25, 39.80, 39.72, 28.63, 26.78, 26.73, 26.67, 25.66, 17.66, 16.21, 16.01.

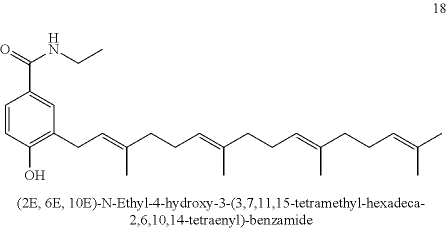

To a solution of (2E,6E,10E)-4-Hydroxy-3-(3,7,11,15-tetramethyl-hexadeca-2,6,10,14-tetraenyl)-benzoic acid (100 mg, 0.25 mmol) in dichloromethane (2 mL) was added N-(3-Dimethylaminopropyl)-W-ethylcarbodiimide (71 mg, 0.37 mmol) and 1-Hydroxibenzotriazol (50 mg, 0.37 mmol) and the reaction was stirred for one hour. Ethylamine (0.15 mL, 0.3 mmol) was then added and the solution was left to stir for further three hours at room temperature. Water was added (25 mL) and the mixture was extracted with dichloromethane (50 mL). The organic layer was washed with brine (25 mL) and the solvent evaporated to give a clear brown oil. The purification by column chromatography, eluent Hexane/Ethyl Acetate (2:1). (67%)

¹H NMR (400 MHz, CDCl₃δ ppm 7.54 (d, J=2.20 Hz, 1H), 7.50 (dd, J=8.31, 2.28 Hz, 1H), 6.84 (d, J=8.32 Hz, 1H), 6.69 (s, 1H), 6.05 (t, J=5.35 Hz, 1H), 5.32 (dt, J=7.14, 1.06 Hz, 1H), 5.15-5.04 (m, 3H), 3.47 (dq, J=7.24, 5.72 Hz, 2H), 3.39 (d, J=7.14 Hz, 2H), 2.18-1.92 (m, 12H), 1.76 (s, 3H), 1.68 (d, J=0.98 Hz, 3H), 1.59 (s, 9H), 1.23 (t, J=7.26 Hz, 3H).

¹³C NMR (100 MHz, CDCl₃δ ppm 167.62, 157.79, 138.63, 135.50, 134.91, 131.22, 128.88, 127.30, 126.47, 126.38, 124.39, 124.22, 123.72, 121.22, 115.57, 39.71, 39.67, 34.92, 29.44, 26.77, 26.62, 26.54, 25.66, 17.66, 16.30, 16.03, 15.99, 14.93

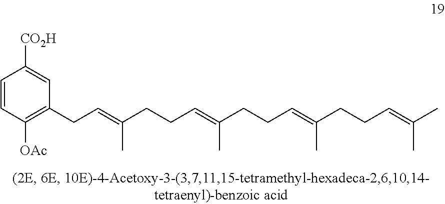

To a pale yellow solution of (2E,6E,10E)-4-Hydroxy-3-(3,7,11,15-tetramethyl-hexadeca-2,6,10,14-tetraenyl)-benzoic acid (100 mg, 0.25 mmol) in anhydrous pyridine, 3 mL) was added Acetic anhydride (0.023 mL, 0.25 mmol) at 0° C. No product was observed at 0° C. after 15 minutes, neither after 1.5 hours. More Acetic anhydride (0.03 mL) was added at 0° C. and the reaction was left to stir at room temperature overnight. The solvent was evaporated to dryness. (73.5%).

¹H NMR (400 MHz, CDCl₃δ ppm 8.02 (d, 1H), 7.99 (dd, J=8.39 Hz, 1H), 7.14 (d, J=8.34 Hz, 1H), 5.26 (dd, J=7.20, 6.14 Hz, 1H), 5.11 (ft, J=8.34, 4.22 Hz, 3H), 3.31 (d, J=7.10 Hz, 2H), 2.33 (s, 3H), 2.20-1.91 (m, 12H), 1.72 (s, 3H), 1.68 (d, J=0.84 Hz, 3H), 1.61 (s, 3H), 1.59 (s, 6H)

¹³C NMR (100 MHz, CDCl₃δ ppm 168.69, 153.28, 137.68, 135.25, 134.86, 134.08, 132.35, 131.17, 129.31, 124.41, 124.24, 123.91, 122.53, 120.63, 39.68, 28.68, 26.77, 26.65, 26.59, 25.64, 20.85, 17.64, 16.26, 16.00, 15.97.

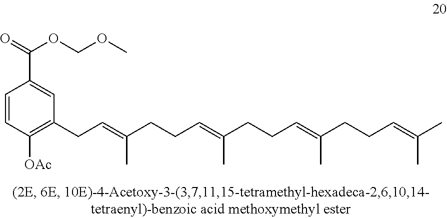

To a solution of (2E,6E,10E)-4-Acetoxy-3-(3,7,11,15-tetramethyl-hexadeca-2,6,10,14-tetraenyl)-benzoic acid methoxymethyl ester (110 mg, 0.24 mmol) in Tetrahydrofuran (1.5 mL) was added Diisopropyl ethylamine (37 mg, 0.29 mmol). To the resulting mixture was added Methyl chloromethyl ether (0.02 mL, 0.29 mmol) at 0° C. and the mixture stirred for 3.5 hours. Diethyl ether (50 mL) was added and the mixture was washed with water (25 mL), 0.1N HCl (10 mL), and brine to give a pale yellow oil. The oil product was purified by radial chromatography employing Hexane/Ethyl acetate (20:1) giving 90 mg of a colourless oil.

¹H NMR (400 MHz, CDCl₃δ ppm 7.98 (d, J=1.97 Hz, 1H), 7.95 (dd, J=8.36, 2.16 Hz, 1H), 7.12 (d, J=8.35 Hz, 1H), 5.47 (s, 2H), 5.28-5.20 (m, 1H), 5.15-5.06 (m, 3H), 3.53 (s, 3H), 3.30 (d, J=7.11 Hz, 2H), 2.32 (s, 3H), 2.17-1.91 (m, 12H), 1.71 (d, J=0.59 Hz, 3H), 1.68 (d, J=1.00 Hz, 3H), 1.60 (s, 6H), 1.59 (d, J=0.91 Hz, 3H)

¹³C NMR (100 MHz, CDCl₃δ ppm 168.70, 165.43, 152.87, 137.58, 135.24, 134.88, 134.03, 131.91, 131.19, 128.78, 127.65, 124.39, 124.21, 123.90, 122.48, 120.69, 90.95, 57.66, 39.69, 28.74, 26.77, 26.65, 25.65, 20.84, 17.64, 16.28, 15.99.

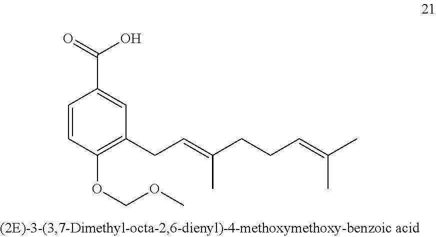

To a solution of the (2E)-3-(3,7-dimethyl-octa-2,6-dienyl)-4-methoxymethoxy-benzaldehyde (150 mg; 0,496 mmoles) in a mixture of THF/water (2.5 ml/0.5 ml) and 2-methyl-2-butene (0.05 ml), sodium dihydrogen phosphate (164 mg; 1.19 mmoles) was added. Then, sodium chlorite (140 mg; 1.24 mmoles) was added and the mixture is stirred during 4 hours at room temperature. The mixture is extracted with CH₂Cl₂ (2×25 ml) and dried over sodium sulfate. A purification using a chromatographic column is performed, using as eluent a mixture of ethyl acetate/hexane in a 1:2 ratio.

¹H NMR (400 MHz, CDCl₃ δppm 7.95 (dd; 1H; J=2.2 Hz; J=8.4 Hz) 7.92 (d; 1H; J=2.3 Hz) 7.10 (d; 1H; J=8.4 Hz) 5.32 (dt; 1H; J=1.1 Hz; J=7.3 Hz) 5.28 (s; 2H) 5.11 (dt; 1H; J=1.3 Hz; J=6.7 Hz) 3.49 (s; 3H) 3.38 (d; 2H; J=7.3 Hz) 2.07 (m; 4H) 1.73 (d; 3H; J=0.8 Hz) 1.67 (d; 3H; J=1.0 Hz) 1.60 (s; 3H)

¹³C NMR (100 MHz, CDCl₃) δppm 171.66; 159.33; 136.75; 131.77; 131.48; 130.82; 129.96; 124.15; 122.26; 121.54; 112.83; 93.97; 56.20; 39.75; 28.51; 26.65; 25.64; 17.67; 16.14.

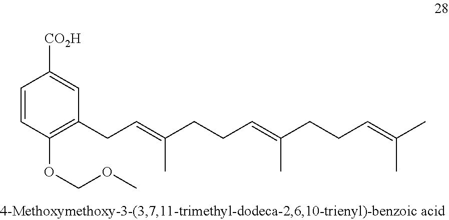

Following a similar reaction strategy as in the preparation of compound 21, compound 28 was obtained by oxidation of (2E,6E)-4-methoxymethoxy-3-(3,7,11-trimethyl-dodeca-2,6,10-trienyl)-benzaldehyde.

1H NMR (400 MHz, CDCl₃) δ ppm 7.93 (dd, J=1.2 Hz, J=8.7 Hz, 1H), 7.92 (d, J=9.26 Hz, 1H), 7.10 (d, J=8.42 Hz, 1H), 5.32 (t, J=7.21 Hz, 1H), 5.28 (s, 2H), 5.18-5.04 (m, 2H), 3.49 (s, 3H), 3.38 (d, J=7.32 Hz, 2H), 2.24-1.87 (m, 8H), 1.73 (s, 3H), 1.67 (s, 3H), 1.59 (s, 6H)

1H NMR (400 MHz, CDCl₃) δ ppm 171.12, 159.31, 136.78, 135.10, 131.78, 131.23, 130.82, 129.96, 124.38, 124.05, 122.19, 121.54, 112.84, 93.97, 56.20, 39.77, 39.68, 28.54, 26.74, 26.61, 25.67, 17.66, 16.18, 15.99.

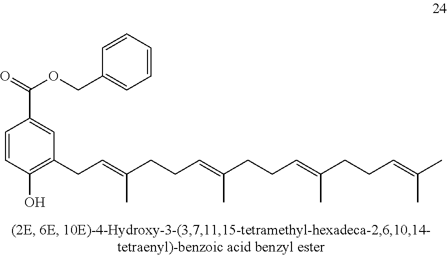

Benzyl bromide (43 mg, 0.25 mmol) was added portion wise to a suspension of (2E,6E,10E)-4-Hydroxy-3-(3,7,11,15-tetramethyl-hexadeca-2,6,10,14-tetraenyl)-benzoic acid (100 mg, 0.25 mmol) and K₂CO₃ (34 mg, 0.25 mmol) in DMF (1.2 mL), and the mixture was stirred for four hours. After two hours, the amber solution with K₂CO₃ in suspension turned to a colourless solution with a white suspension. Water was added and the resulting mixture was extracted with ethyl ether (25 mL). The organic phase was washed eight times with water (10 mL) and one time with brine, and the solvent evaporated under vacuum. The purification was performed by radial chromatography employing a mixture of Hexane/Ethyl acetate (10:1) as eluent, giving 44 mg (36%) of the desired product as amber syrup.

¹H NMR (400 MHz, CDCl₃, δ ppm) 7.89-7.85 (m, 2H), 7.46-7.30 (m, 5H), 6.83 (d, J=8.89 Hz, 1H), 5.86 (s, 1H), 5.35 (s, 2H), 5.32 (m, 1H), 5.14-5.07 (m, 3H), 3.40 (d, J=7.15 Hz, 2H), 2.18-1.94 (m, 14H), 1.79 (s, 3H), 1.69 (s, 3H), 1.62-1.59 (m, 9H).

¹³C NMR (100 MHz, CDCl₃, δ ppm) 166.68, 159.19, 139.55, 136.56, 135.86, 135.14, 132.26, 131.44, 130.10, 128.76, 128.32, 128.27, 127.01, 124.63, 124.46, 123.81, 122.67, 121.154, 115.880, 66.609, 39.941, 39.921, 39.881, 29.901, 27.008, 26.832, 26.643, 25.894, 17.892, 16.547, 16.277, 16.221.

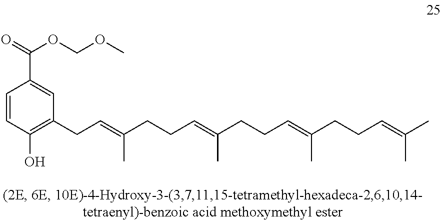

To a solution of (2E,6E,10E)-4-Hydroxy-3-(3,7,11,15-tetramethyl-hexadeca-2,6,10,14-tetraenyl)-benzoic acid (100 mg, 0.25 mmol) and di-isopropylethyl amine (0.05 mL, 0.30 mmol) in THF (1.2 mL), was added chloro-methoxy methane (0.02 mL, 0.30 mmol) and the mixture was stirred for three hours. Another portion of chloro-methoxy methane (0.02 mL, 0.30 mmol) was again added at 0° C., and the stirring was continued for further 1.5 hours. Diethyl ether (25 mL) was added and the resulting mixture was washed with water (15 mL×2), saturated NaCl solution (10 mL×2), dried (Na₂SO₄), and the solvent evaporated under vacuum. The resulting solid was purified by radial chromatography employing a mixture of Hexane/Ethyl Acetate (from 10:1 to 1:1) as eluent, giving 82 mg (61%) of the desired product as a pale yellow solid.

¹H NMR (400 MHz, CDCl₃ δ ppm) 7.88-7.84 (m, 2H), 6.84 (d, J=8.21 Hz, 1H), 5.46 (s, 2H), 5.34 (dt, J=7.15, 7.14, 1.07 Hz, 1H), 5.13-5.07 (m, 3H), 3.54 (s, 3H), 3.41 (d, J=7.17 Hz, 2H), 2.18-1.92 (m, 14H), 1.78 (s, 3H), 1.68 (s, 3H), 1.60 (s, 6H).

¹³C NMR (100 MHz, CDCl₃, δ ppm) 166.109, 159.194, 139.104, 135.558, 134.877, 132.083, 131.176, 129.922, 127.033, 124.379, 124.200, 123.605, 121.936, 120.913, 115.595, 90.626, 57.565, 39.686, 39.626, 29.465, 26.747, 26.576, 26.433, 25.629, 17.626, 16.268, 16.007, 15.955.

Compounds 23, 26 and 27 were prepared according to the following general reaction scheme:

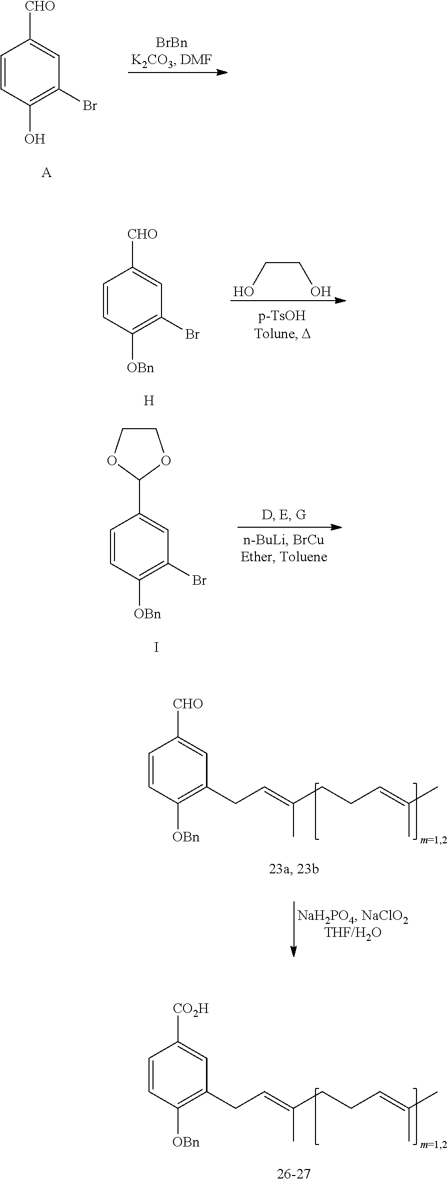

Benzyl ether H was prepared by protection of 3-bromo-4-hydroxybenzaldehyde in the presence of potassium carbonate (yield 91%). Then, reaction of the aldehyde with ethyleneglycol and a catalytic amount of p-toluenesulfonic acid afforded acetal I in moderate yield (Ling. et al., J. Org. Chem. 2001, 66, 8843). Further alkylation of the aryl bromide was achieved by addition of n-BuLi followed by copper bromide and then by the corresponding prenylic bromide, thus obtaining the corresponding aldehydes (23a and 23b). Further oxidation in the presence of NaH₂PO₄ and NaCl₂O₂ gave rise to acids 26 and 27 in high yield.

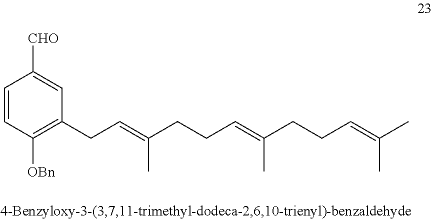

¹H NMR (400 MHz, CDCl₃ [symbol] ) ppm 9.86 (s, 1H), 7.71 (s, 1H), 7.70 (dd, J=6.96, 2.10 Hz, 1H), 7.46-7.31 (m, 5H), 7.00 (d, J=9.06 Hz, 1H), 5.39-5.27 (m, 1H), 5.18 (s, 2H), 5.15-5.03 (m, 2H), 3.43 (d, J=7.28 Hz, 2H), 2.23-1.81 (m, 8H), 1.67 (s, 6H), 1.59 (s, 3H), 1.58 (s, 3H).

¹³C NMR (100 MHz, CDCl₃ δ) ppm 191.37, 161.73, 137.41, 136.51, 135.33, 131.62, 131.48, 130.93, 130.54, 130.02, 128.86, 128.35, 127.42, 124.57, 124.29, 121.42, 111.42, 70.40, 40.00, 39.92, 28.71, 26.95, 26.82, 25.91, 17.91, 16.41, 16.24.

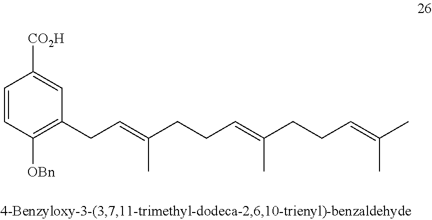

¹H-NMR (25° C., CDCl₃, 400 MHz, ppm) 7.95 (dd, 1H, J=1.2 Hz, J=8.5 Hz); 7.92 (d, 1H, J=1.9 Hz); 7.38 (m, 5H); 6.94 (d, 1H, J=8.5 Hz); 5.34 (t, 1H, J=7.2 Hz); 5.17 (s, 2H); 5.11 (m, 2H); 3.42 (d, 2H, J=7.2 Hz); 2.04 (m, 8H); 1.67 (s, 6H); 1.59 (s, 3H); 1.59 (s, 3H).

¹³C-NMR (25° C.; CDCl₃; 100 MHz; ppm) 177.7; 160.8; 136.8; 136.5; 135.0; 131.6; 131.2; 130.6; 130.1; 128.5; 128.0; 127.1; 124.4; 124.1; 121.5; 121.4; 110.8; 70.0; 39.7; 39.6; 28.5; 26.7; 26.6; 25.6; 17.6; 16.1; 16.0.

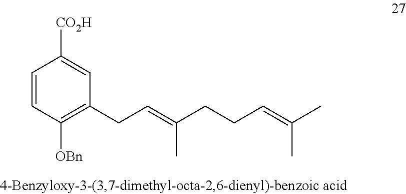

¹H-NMR (25° C., CDCl₃, 400 MHz, ppm) 7.95 (dd, 1H, J=1.2 Hz, J=8.7 Hz); 7.92 (d, J=1.9 Hz, 1H); 7.39 (m, 5H); 6.94 (d, 1H, J=8.5 Hz); 5.34 (t, 1H, J=7.4 Hz); 5.17 (s, 2H); 5.11 (t, 1H, J=6.7 Hz); 3.41 (d, 2H, J=7.2 Hz); 2.08 (m, 4H); 1.67 (s, 6H); 1.60 (m, 3H)

¹³C-NMR (25° C., CDCl₃, 100 MHz, ppm) 171.2; 160.8; 136.8; 136.5; 131.6; 131.4; 130.6; 130.1; 128.5; 128.0; 127.2; 124.2; 121.5; 121.4; 110.8; 70.0; 39.7; 28.5; 26.6; 25.6; 17.6; 16.1.

### Biological Methods

### Example 4

### BACE Assay

The aim of this assay is to determine if a compound, either synthetic or of marine origin, is a BACE-1 inhibitor, to avoid the formation of Aβ. This assay is based on FRET technology (Fluorescence Resonance Energy Transfer). FRET is used to measure cleavage of a peptide substrate, among other uses. The peptide substrate shows two fluorophores, a fluorescence donor and a quenching acceptor. The distance between these two fluorophores has been selected so that upon light excitation, the donor fluorescence energy is significantly quenched by the acceptor. When a substrate peptide cleavage occurs, the energy balance is broken and all the donor fluorescence can be observed. The increase in fluorescence is linearly related to the rate of proteolysis (Gordon, G W et al., 1998). In this assay the reaction occurs between an enzyme, purified BACE-1, and a fluorogenic peptidic substrate who present the “Swedish mutation”. The peptide cleavage by BACE-1 produces fluorescence energy and enzymatic activity can be quantified.

The reagents which are used in this assay are the following:

rhBACE-1 β-Secretase recombinant human (R&D Systems. Ref. 931-AS).

Fluorogenic Peptide Substrate IV (R&D Systems. Ref. ES004).

Beta-SECRETASE INHIBITOR H-4848. (BACHEM. Ref. H-4848.0001).

Sodium acetate.

The assay is carried out in a 96 wells microplate. The final concentration of substrate is 3.5 μM per well, and the enzyme concentration is 0.5 μg/ml. The final volume of the assay is 100 μl per well and all reagents are diluted in Reaction Buffer.

The compounds are tested at a concentration of 10⁻⁵ and 10⁻⁶ M. The control in the assay is the commercial inhibitor β-Secretase inhibitor H-4848 from BACHEM, which is tested at 300 nM. All the samples and controls are studied by duplicate.

The plate is mixed gently and changes in the fluorescence are measured using a fluorimeter plate reader, with 320 nm excitation filter and 405 nm emission filter. The temperature should be preferably maintained between 25 and 30° C. Measurements are carried out every ten minutes during an hour. The first measure is subtracted from the last to calculate the fluorescence increase, evaluating the enzymatic activity. The 100% activity is calculated as the mean of the results of wells without sample or inhibitor.

In the cases where abnormal effects in fluorescence were detected, BACE inhibition activity was assayed using BACE-1 (beta-Secretase) FRET ASSAY KIT (Invitrogen, Ref. P2985). Fluorescence was measured with a fluorimeter plate reader, with 544 nm excitation filter and 580 nm emission filter.

Further information regarding this assay may be found in the following references, which are incorporated by reference into the present application:
Andrau, D et al; “BACE1- and BACE2-expressing human cells: characterization of beta-Amyloid precursor protein-derived catabolites, design of a novel fluorimetric assay, and identification of new in vitro inhibitors”. J Biol Chem. 2003 Jul. 11; 278(28):25859-66.
Gordon, G W et al; “Quantitative fluorescence resonance energy transfer measurements using fluorescence microscopy.” Biophys J. 1998 May; 74:2702-13.

The compounds of formula (I) of the present invention where submitted to the above indicated assay, in order to determine their BACE activity inhibition. The results are indicated in Table I, in percentage of the enzyme activity.

TABLE I

| | % BACE Activity | | |
|---|---|---|---|
| | 1 μM | 10 μM | IC50 |
| Compound 1 | 45 ± 21 | 0 | / |
| Compound 2 | 100 | 13 ± 14 | / |
| Compound 6 | 100 | 100 | / |
| Compound 17 | 100 | 100 | / |
| Compound 24 | 100 | 100 | / |
| Compound 18 | 100 | 100 | / |
| Compound 19 | 87 ± 14.5 | 17.5 ± 0.7 | 20.5 ± 2.8 μM |
| Compound 25 | 100 | 100 | / |
| Compound 20 | 100 | 100 | / |
| Compound 26 | 100 | 100 | / |
| Compound 27 | 34 ± 27 | 0 ± 0 | / |
| Compound 12 | 57 ± 2 | 41 ± 4 | / |
| Compound 9 | 94 ± 22 | 60 ± 16 | 3.7 ± 2.5 × 10−5M |
| Compound 28 | 87 ± 10 | 33 ± 7 | / |
| Compound 15 | 100 | 5 ± 9 | / |
| Compound 8 | 100 | 42 ± 5 | / |
| Compound 11 | 91 ± 6 | 44 ± 12 | / |
| Compound 14 | 100 | 84 ± 6 | / |
| Compound 21 | 88 ± 9 | 27 ± 3 | / |
| Compound 23 | 99 ± 2 | 88 ± 13 | / |
| Compound 13 | 100 | 88 ± 8.5 | / |

### Example 5

### GSK-3 Beta Inhibition Assay

The GSK-3 beta activity of the compounds of formula (I) according to the present invention was determined by incubation of a mixture of recombinant human GSK-3 enzyme, a phosphate source and GSK-3 substrate in the presence and in the absence of the corresponding test compound, and by measuring the GSK-3 activity of this mixture. The compounds where tested at final concentrations of 25 and 50 μM.

Recombinant human glycogen synthase kinase 3 beta was assayed in MOPS 11 mM, pH 7.4, EDTA 0.2 mM, EGTA 1.25 mM, MgCl₂ 26.25 mM and sodium orthovanadate 0.25 mM in the presence of 62.5 μM of Phospho-Glycogen Synthase Peptide-2 (GS-2), 0.5 μCi gamma-³³P-ATP and unlabelled ATP at a final concentration of 12.5 μM. The final assay volume was 20 μl. After incubation for 30 minutes at 30° C., 15 μl aliquots were spotted onto P81 phosphocellulose papers. Filters were washed four times for at least 10 minutes each and counted with 1.5 ml of scintillation cocktail in a scintillation counter.

The compounds of formula (I) of the present invention where submitted to the above indicated assay, in order to determine their GSK-3 inhibition activity. The results are indicated in Table II, in percentage of the enzyme activity.

TABLE II

| | % Act. GSK-3 beta | | | |
|---|---|---|---|---|
| | 25 μM | 50 μM | IC50 | |
| Compound 1 | 5.43 | 2.82 | 4.42 | μM |
| Compound 2 | 37.74 | 5.04 | 12.5 | μM |
| Compound 3 | 52.86 | 4.19 | 55.82 | μM |
| Compound 4 | 17.79 | 17.70 | 9.97 | μM |
| Compound 5 | / | 70.85 | / | |
| Compound 6 | 71.56 | 78.03 | / | |
| Compound 17 | 71.93 | 52.11 | / | |
| Compound 24 | 49.72 | 22.78 | / | |
| Compound 18 | 32.81 | 13.34 | / | |
| Compound 19 | 10.68 | 4.58 | 2.77 | μM |
| Compound 25 | 9.18 | 4.5 | 21.96 | μM |
| Compound 20 | 51.05 | 17.67 | / | |
| Compound 26 | 71.41 | 40.96 | / | |
| Compound 27 | 85.11 | 65.85 | / | |
| Compound 12 | 9.26 | 0.95 | 7.87 | μM |
| Compound 9 | 61.3 | 5.75 | / | |
| Compound 28 | 17.05 | 2.44 | 17.96 | μM |
| Compound 15 | 24.56 | 3.27 | 10.97 | μM |
| Compound 8 | 33.54 | 3.08 | 16 | μM |
| Compound 11 | 12.1 | 1.07 | 8.6 | μM |
| Compound 14 | 75.7 | 60.3 | / | |
| Compound 21 | 100.5 | 79.9 | / | |
| Compound 23 | 2.22 | 2.01 | / | |
| Compound 13 | 52.01 | 4.71 | 17.42 | μM |

1. A compound of general formula (I) 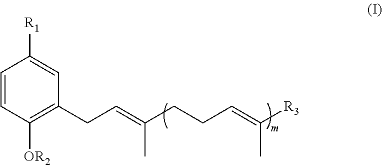
wherein
m is an integer selected from 0, 1, 2, 3, 4, and 5;
R₁ is selected from —C(═O)OR₄, —CHO and —CONH—R₅,
wherein R₄ is selected from hydrogen, —CH₂-Ph, —CH₂—O—CH₃,
and R₅ is C₁-C₆ alkyl,
R₂ is selected from hydrogen, phenyl, benzyl, —COR₆ and —CH₂—O—CH₃,
wherein R₆ is selected from hydrogen and C₁-C₆ alkyl,
R₃ is selected from —CH₃ and 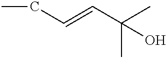
and salts, preferably any pharmaceutically acceptable salts, solvates and prodrugs thereof.

2. A compound according to claim 1, wherein m is selected from 0, 1, 2 and 3.

3. A compound according to claim 1, wherein R₁ is —C(═O)OR₄, R₄ being selected from hydrogen, —CH₂—O—CH₃ and —CH₂-Ph.

4. A compound according to claim 3, wherein R₄ is selected from —CH₂—O—CH₃ and —CH₂-Ph.

5. A compound according to claim 1, wherein R₁ is —CONH—R₅, R₅ being selected from methyl and ethyl.

6. A compound according to claim 1, wherein R₂ is selected from hydrogen, benzyl, —COCH₃ and —CH₂—O—CH₃.

7. A compound according to claim 6, wherein R₂ is selected from benzyl and —COCH₃.

8. A compound according to claim 1, wherein R₃ is

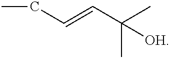

9. A compound according to claim 1, wherein m is an integer selected from 1, 2, 3, 4, and 5; R₁ is —CHO and R₂ is —CH₂—O—CH₃.

10. A compound according to claim 1, wherein m is an integer selected from 0, 1 and 2; R₁ is —C(═O)OH and R₂ is CH₂—O—CH₃.

11. A compound according to claim 1, wherein m is an integer selected from 2, 3, 4, and 5; R₁ is —CHO and R₂ is hydrogen.

12. A compound according to claim 1, wherein m is an integer selected from 2, 4 and 5; R₁ is —C(═O)OH and R₂ is hydrogen.

13. A compound according to claim 1, selected from:

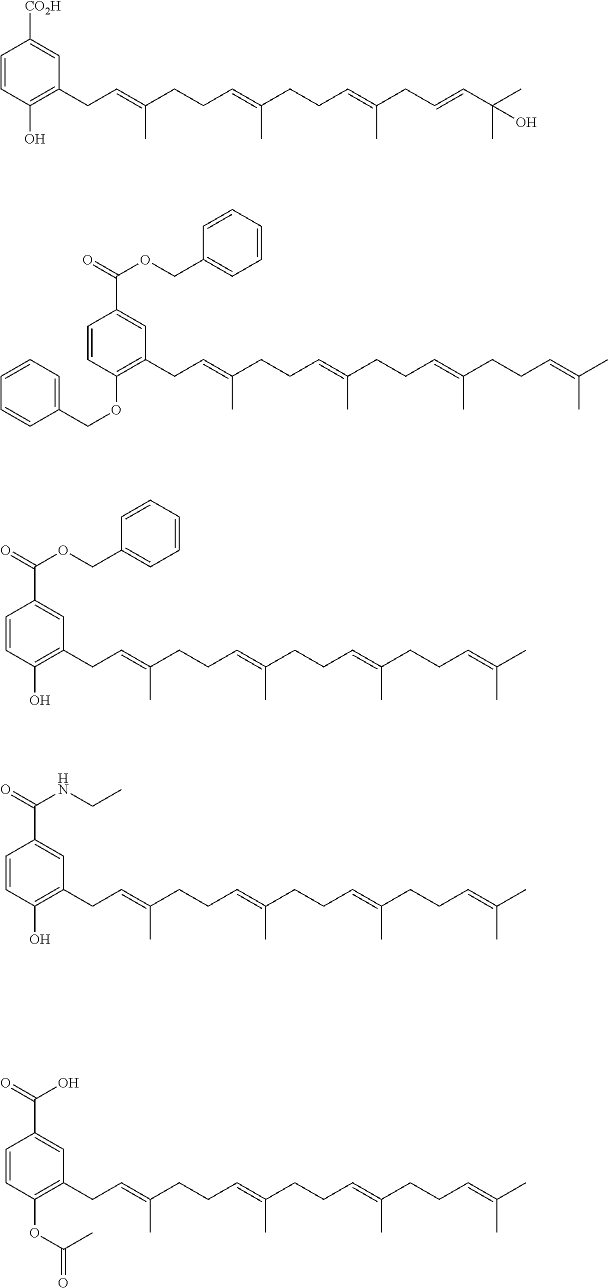

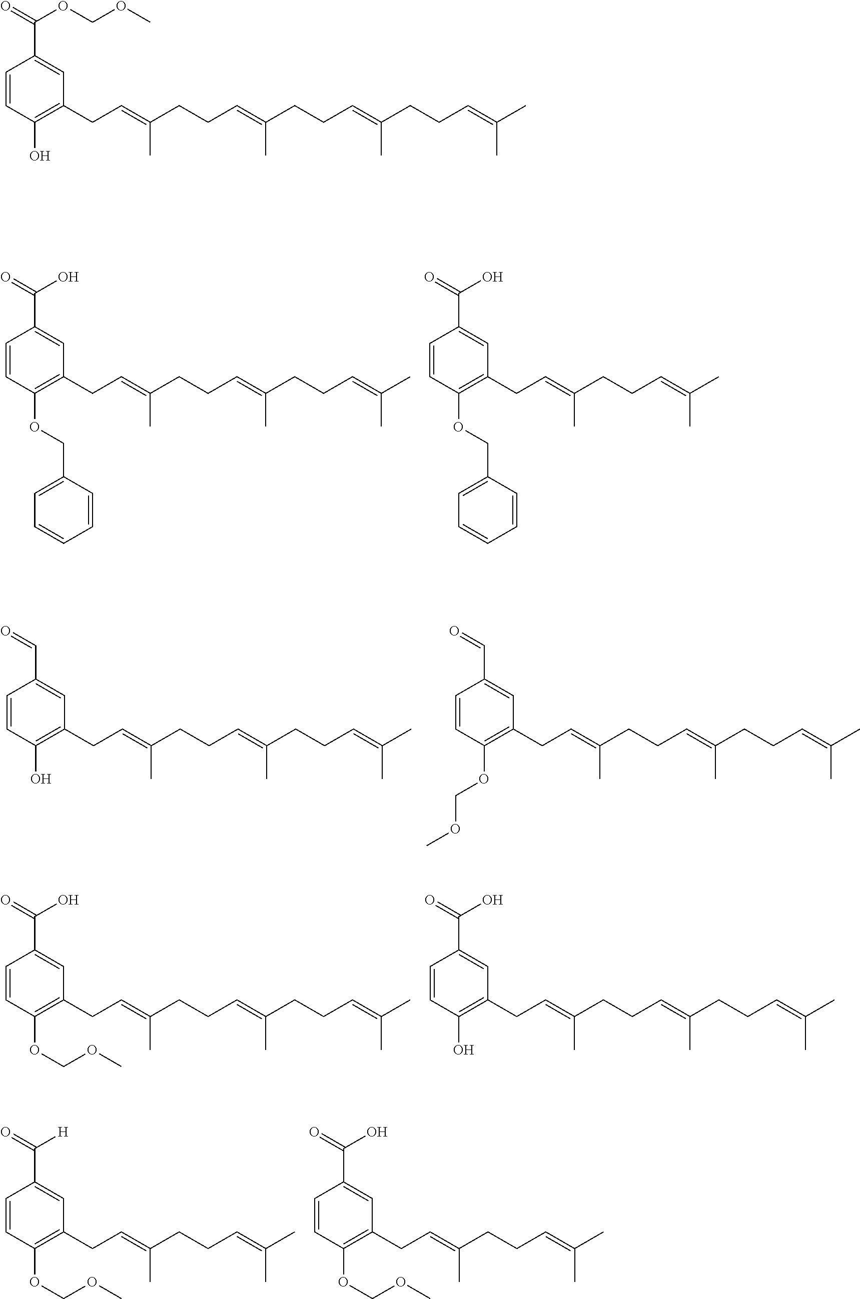

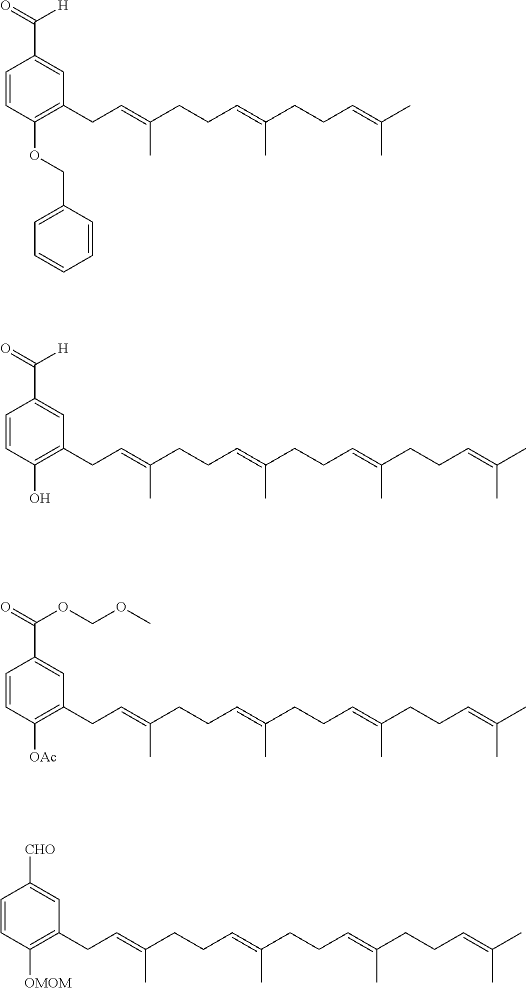

14. (canceled)

15. A pharmaceutical composition comprising the compound of formula (I) as defined in claim 1, or salts, solvates or prodrugs thereof, and at least one pharmaceutically acceptable carrier, adjuvant and/or vehicle.

16. Method of treating and/or preventing a cognitive, neurodegenerative or neuronal disease or disorder, which method comprises administering to a patient in need of such a treatment a therapeutically effective amount of at least one compound of formula (I*). 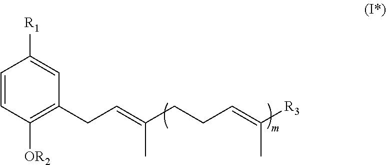
wherein
m is an integer selected from 0, 1, 2, 3, 4, and 5;
R₁ is selected from C₁-C₁₂ alkoxy, —CH₂—O—CH₃, —OH, —C(═O)OR₄, —CHO and —CONH—R₅,
wherein R₄ is selected from hydrogen, C₁-C₆ alkyl, —CH₂-Ph, —CH₂—O—CH₃,
and R₅ is C₁-C₆ alkyl,
R₂ is selected from hydrogen, phenyl, benzyl, —COR₆, C₁-C₆ alkyl and —CH₂—O—CH₃,
wherein R₆ is selected from hydrogen and C₁-C₆ alkyl,
R₃ is selected from —CH₃ and 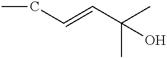
and salts, preferably any pharmaceutically acceptable salts, solvates and prodrugs thereof.

17. Method according to claim 16, wherein the cognitive, neurodegenerative or neuronal disease or disorder is selected from chronic neurodegenerative conditions including dementias such as Alzheimer's disease, Parkinson's disease, progressive supranuclear palsy, subacute sclerosing panencephalitic parkinsonism, postencephalitic parkinsonism, pugilistic encephalitis, guam parkinsonism-dementia complex, Pick's disease, corticobasal degeneration, frontotemporal dementia, Huntington's Disease, AIDS associated dementia, amyotrophic lateral sclerosis, multiple sclerosis and neurotraumatic diseases such as acute stroke, epilepsy, mood disorders such as depression, schizophrenia and bipolar disorders, promotion of functional recovery post stroke, cerebral bleeding, such as cerebral bleeding due to solitary cerebral amyloid angiopathy, mild cognitive impairment, Hereditary Cerebral Hemorrhage with Amyloidosis of the Dutch-Type, cerebral Amyloid angiopathy, ischaemia, brain injury, especially traumatic brain injury, Down's syndrome, Lewy body disease, inflammation and chronic inflammatory diseases.

18-20. (canceled)

21. A compound according to claim 2, wherein R₁ is —C(═O)OR₄, R₄ being selected from hydrogen, —CH₂—O—CH₃ and —CH₂-Ph.

22. A compound according to claim 3, wherein R₂ is selected from hydrogen, benzyl, —COCH₃ and —CH₂—O—CH₃.

23. A compound according to claim 21, wherein R₂ is selected from hydrogen, benzyl, —COCH₃ and —CH₂—O—CH₃.

24. A compound according to claim 22, wherein R₂ is selected from benzyl and —COCH₃.

25. A compound according to claim 23, wherein R₂ is selected from benzyl and —COCH₃.
